(19) United States
(12) Patent Application Publication
(10) Pub. No.: US 2017/0362585 A1
(43) Pub. Date: Dec. 21, 2017

(54) METHODS AND APPARATUS FOR X-GENETICS

(71) Applicants: Ge Wang, Loudonville, NY (US); Matthew Webber Getzin, Troy, NY (US); Chunyu Wang, Latham, NY (US); Jian Kang, Troy, NY (US)

(72) Inventors: Ge Wang, Loudonville, NY (US); Matthew Webber Getzin, Troy, NY (US); Chunyu Wang, Latham, NY (US); Jian Kang, Troy, NY (US)

(73) Assignee: RENSSELAER POLYTECHNIC INSTITUTE, Troy, NY (US)

(21) Appl. No.: 15/624,492

(22) Filed: Jun. 15, 2017

Related U.S. Application Data
(60) Provisional application No. 62/350,364, filed on Jun. 15, 2016.
(60) Provisional application No. 62/420,005, filed on Nov. 10, 2016.

(51) Int. Cl. C12N 13/00; G06T 11/00; A61N 5/06; G06N 3/08; C12N 5/0793; A61N 5/10

(52) U.S. Cl. CPC C12N 13/00; C12N 5/0619; A61N 5/10; A61N 5/0622; G06N 3/08; G06T 11/006; C12N 2510/00; A61N 2005/0663; A61N 2005/0651; G06T 2211/421

(57) ABSTRACT

Methods and systems of using X-ray radiation to irradiate X-ray sensitive biomolecules to allow for specific control over the behavior of cells via the X-ray irradiation are provided. The systems and methods are influenced by the field of optogenetics, which uses visible light instead of X-ray radiation. X-ray stimulation penetrates both bone and soft tissue with very little attenuation and can be performed without any physical contact with the sample. Image reconstruction methods using deep learning are also provided. A deep learning algorithm can be used to obtain a reconstructed image from raw data obtained via medical imaging, either with or without first performing a conventional algorithm.

### CROSS REFERENCE TO RELATED APPLICATIONS

This application claims the benefit of U.S. Provisional Patent Application Ser. No. 62/350,364, filed Jun. 15, 2016, and U.S. Provisional Application Ser. No. 62/420,005, filed Nov. 10, 2016, the disclosures of which are hereby incorporated by reference in their entirety, including any figures, tables, and drawings.

### BACKGROUND

Developing multidisciplinary approaches for molecular imaging, neuroscience, and interventional tools (e.g., aimed at the nervous system) is very important and is even an emphasis of the NIH roadmap and BRIAN initiative. In this context, optogenetics has recently become a critical technique for studying brain circuits and functions. In addition, it has potential for treating neurological disorders such as depression, Alzheimer's, and Parkinson's diseases. A major limitation of optogenetics is that light cannot penetrate deeply into biological tissue. Indeed, part of the skull has to be surgically removed for inserting a light probe near an area of interest, and only a small depth (less than a few millimeters) of the cortex is available for stimulation from the surgical window as light is quickly attenuated by the brain tissue.

### BRIEF SUMMARY

Embodiments of the subject invention provide methods and systems of using X-ray radiation to irradiate X-ray sensitive biomolecular mechanisms to allow for specific control over the behavior of cells via the X-ray irradiation. The systems and methods are influenced by the field of optogenetics, which uses visible light (and not X-ray radiation). X-ray stimulation, as used with embodiments of the subject invention, offers the distinct advantage (over optogenetics) of penetrating both bone and soft tissue with very little attenuation, thereby allowing for much less invasive stimulation and the ability to stimulate deep tissues that visible light cannot reach. This can be referred to as “X-Genetics”.

Embodiments of the subject invention also provide image reconstruction methods using deep learning. A deep learning algorithm and/or deep neural network can be used to obtain a reconstructed image from raw data (e.g., features) obtained with medical imaging (e.g., CT, MM, X-ray). In a specific embodiment, a conventional (i.e., non-deep-learning) reconstruction algorithm can be used on the raw imaging data to obtain an initial image, and then a deep learning algorithm and/or deep neural network can be used on the initial image to obtain a reconstructed image.

In an embodiment, a method of controlling the behavior of a cell (e.g., a neuron) in a sample can comprise providing X-ray radiation to an X-ray sensitive biomolecule within the sample to stimulate the X-ray sensitive biomolecule. The stimulation of the X-ray sensitive biomolecule can causes a change in the membrane potential of the neuron, thereby changing the behavior of the neuron.

In another embodiment, a method of reconstructing an image from raw data obtained by a medical imaging process can comprise performing at least one algorithm on the raw data to obtain a reconstructed image, wherein the at least one algorithm comprises a deep learning algorithm. The deep learning algorithm can be performed directly on the raw data to obtain the reconstructed image, or a conventional algorithm can be performed first on the raw data to obtain an initial image, followed by performing the deep learning algorithm on the initial image to obtain the reconstructed image.

### BRIEF DESCRIPTION OF DRAWINGS

FIG. 1 shows a 3D rendering of a system according to an embodiment of the subject invention. A field-emission X-ray source can be used to generate X-ray radiation with a diagnostic range of voltage and current settings and an instantaneous X-ray radiation control (e.g., <<1 ms switching time), which can will allow a direct, rapid, and precise generation of periodic and non-periodic X-ray pulses of various shapes, providing great flexibility in testing dynamic physiological interactions between a sample (e.g., a retina of an organism) and X-rays. Also, an X-ray filter can be designed and used to determine the relationship between the X-ray sensitivity of a protein (e.g., an opsin) and the X-ray wavelength (energy) of the stimulation pulses. The X-ray filter can be implemented utilizing optimized combinations of K-edge of various materials (e.g., iodine, barium, gadolinium, ytterbium, tantalum, gold, and Bismuth with their K-edges at 33, 37, 50, 61, 67, 81, and 91 keV, respectively. Finer X-ray wavelength resolution can be achieved via Bragg diffraction on mosaic crystal, from inverse-Compton scattering, or on a synchrotron facility.

FIG. 2 shows a close up of the X-ray source and sample (a frog is depicted), with the inset showing an enlarged version of where the sample can be positioned.

FIG. 3 shows a different angle of the sample positioning from FIGS. 1 and 2.

FIG. 4 shows a flowchart of two different methods to identify X-ray sensitive molecules (e.g., proteins such as opsins). Although FIG. 4 discusses identifying opsins in a frog, this is for exemplary purposes only, and the methods depicted can be used for identification of other types of molecules in other types of samples or organisms.

FIG. 5 shows an image of bacterial rhodopsin purification via a sucrose gradient. The dark band with the arrow pointing to it is the rhodopsin.

FIG. 6 shows a depiction of the structure of different types of opsins.

FIGS. 7A-7H show response curves of differential voltage potential (micro-Volts) versus time (seconds) for visible electroretinography (ERG) measurements from frogs tested. These curves were categorized as “visible responses”. The flattened curves seen from certain frogs (arbitrarily labeled 25, 28, and 31) were a result of unexpected jumps in signal amplitude where the dynamic range of the measured data had been capped.

FIGS. 8A-8H show noise recording curves of differential voltage potential (micro-Volts) versus time (seconds) for visible ERG measurements from frogs tested. These curves were categorized either as steady noise with no response after t=0 or as high amplitude noise. In the cases of consistent, large fluctuations in baseline signal, as seen in certain frogs (arbitrarily labeled 26, 28, and 30), the classification could have missed some visible responses that were buried in noise or could have mistaken noise as a response of some kind.

FIGS. 9A-9D show response curves of differential voltage potential (micro-Volts) versus time (seconds) for visible ERG measurements from frogs tested. These curves show “positive only” X-ray ERG responses. Only half the frogs were measured to have such a response to the X-ray stimuli, and each of the responses occurred after stimulation with a 200 millisecond X-ray pulse.

FIGS. 10A-10C show response curves of differential voltage potential (micro-Volts) versus time (seconds) for visible ERG measurements from frogs tested. These curves show “negative only” X-ray ERG responses. Less than half of the frogs responded to the X-ray stimulation in this way, and even more surprisingly, 8 of 11 of these measured signals were measured when the electrodes were placed on the back of the tested frog.

FIGS. 11A-11C show plots of differential voltage potential (micro-Volts) versus time (seconds). FIG. 11A is for visible, FIG. 11B is for positive-only, and FIG. 11C is for negative-only responses for ERG measurements from tests on frogs. These summarize how full width at half maximum (FWHM) and area under the curve (AUC) were calculated for each of the response types. As seen in the scatter plots, the a-wave of the visible responses is clearly different from the negative responses to X-ray stimuli. The b-wave and the positive X-ray response distributions, on the other hand, are not separable by these metrics.

FIGS. 12A-12H show plots of bootstrap non-parametric density versus different wave parameters (as labeled in the figures). These are comparisons with adjusted p-values.

FIG. 12G, of b-wave FWHM (seconds) has p<0.1, and FIGS. 12A (a-wave time (seconds)), 12B (a-wave magnitude (micro-Volts)), 12C (a-wave FWHM (seconds)), 12D (a-wave AUC (seconds*micro-Volts)), 12F (b-wave magnitude (micro-Volts)), and 12H (b-wave AUC (seconds*micro-Volts)) have significant p values (p<0.05).

FIG. 13A shows a plot of differential voltage potential (micro-Volts) versus time (seconds), illustrating an X-ray induced decrease in visible light ERG for a frog tested (arbitrarily labeled as frog 29). The (blue) curve with the highest peak is the average visible response before X-ray exposure, the (orange) curve with the lowest peak is the noise response to 200-millisecond X-ray stimulation, and the (yellow) curve with the second-highest peak is the average visible response after X-ray stimulation.

FIG. 13B shows a plot of differential voltage potential (micro-Volts) versus time (seconds), illustrating the average responses with the electrodes placed on the back a frog tested (arbitrarily labeled as frog 29). No response to either visible or X-ray stimulation was observed.

FIG. 14A shows a plot of differential voltage potential (micro-Volts) versus time (seconds), illustrating an X-ray induced decrease in visible light ERG for a frog tested (arbitrarily labeled as frog 31). The (blue) curve with the highest peak is the average visible response before X-ray exposure, the (orange) curve with the lowest peak is the noise response to 200-millisecond X-ray stimulation, and the (yellow) curve with the second-highest peak is the average visible response after X-ray stimulation.

FIG. 14B shows a plot of differential voltage potential (micro-Volts) versus time (seconds), illustrating the average responses with the electrodes placed on the back a frog tested (arbitrarily labeled as frog 29). No response to visible stimulation was observed, but a small negative response to X-ray stimulation was observed.

FIG. 15 shows a schematic representation of deep imaging.

FIG. 16 shows a schematic representation of a biological neuron and an artificial neuron.

FIG. 17 shows a schematic representation of a deep network for feature extraction and classification through nonlinear multi-resolution analysis.

FIG. 18 shows a schematic visualization of inner product as a double helix.

FIG. 19 shows a schematic view of imaging that can be achieved with deep imaging.

FIG. 20 shows eight images demonstrating a deep network capable of iterative reconstruction. The image pair in the left-most column are two original phantoms; the image pair in the second-from-the-left column are the simultaneous algebraic reconstruction technique (SART) reconstruction after 20 iterations; the image pair in the second-from-the-right column are the SART reconstruction after 500 iterations; and the image pair in the right-most column are the deep imaging results after starting with the corresponding 20-iteration image (from the second-from-the-left column) as the inputs, which are very close to the 500-iteration images, respectively.

FIG. 21 shows three images demonstrating a deep network capable of sinogram restoration. The first row shows an original image (metal is the small (purple) dot in the upper-left corner) and the associated metal-blocked sinogram. The second and third rows are the original and restored sinograms, respectively, which show the potential of deep learning as a smart interpolator over missing data.

FIGS. 22A-22C show images demonstrating a deep network image reconstruction. FIG. 22A shows the full-dose filtered back-projection image; FIG. 22B shows the quarter-dose filtered back-projection image; and FIG. 22C shows the deep learning reconstruction image using the quarter-dose filtered back-projection image of FIG. 22B as the starting input.

FIG. 23A shows the total absolute area under the curve of a, b, and c waves relative to a response voltage baseline for an experiment performed on frogs.

FIGS. 23B, 23C, and 23D show visible light-induced ERGs before X-ray exposure for three frogs tested, respectively.

FIGS. 23E, 23F, and 23G show X-ray induced response for three frogs tested, respectively.

FIGS. 23H, 23I, and 23J show the visible light-induced ERGs after X-ray exposure for three frogs tested, respectively.

FIG. 23K shows the average and standard deviations for the data in FIGS. 23B-23J.

FIG. 24 shows a schematic view of a mechanism of phototransduction in a rod cell.

### DETAILED DESCRIPTION

Embodiments of the subject invention provide methods and systems of using X-ray radiation to irradiate X-ray sensitive biomolecular mechanisms that can be naturally in cells of an organism (e.g., an animal such as a frog or a human) or genetically introduced into cells to allow for specific control over the behavior of the cell(s) via the X-ray irradiation. The systems and methods are influenced by the field of optogenetics, which uses visible light (and not X-ray radiation). X-ray stimulation, as used with embodiments of the subject invention, offers the distinct advantage (over optogenetics) of penetrating both bone and soft tissue with very little attenuation, thereby allowing for much less invasive stimulation and the ability to stimulate deep tissues that visible light cannot reach. This can be referred to as “X-Genetics”.

Embodiments of the subject invention are related to some aspects of U.S. Patent Application Publication No. 2016/0166852 (Wang et al., “X-Optogenetics/U-Optogenetics”), which is hereby incorporated herein by reference in its entirety. However, whereas U.S. Patent Application Publication No. 2016/0166852 requires providing light-emitting particles such as nanophosphors to a sample before providing X-rays for X-Optogenetics, embodiments of the subject invention can specifically exclude providing any light-emitting particles to a sample or organism (i.e., no light-emitting particles are provided to the sample or organism before X-ray radiation is provided to the sample or organism). X-ray sensitive molecules, such as proteins (e.g., opsins) in an organism or sample can be stimulated with focused X-ray radiation to control the behavior of one or more cells via the X-ray irradiation, similar to how optogenetics controls cells, but far less invasive and allowing for deeper tissues. X-ray radiation can be provided to molecules that are known ahead of time to be X-ray sensitive in an organism or sample. For example, opsins, such as those in the retina of the Northern leopard frog (Rana pipiens) can be provided with X-ray radiation. Opsins in the retina of the Northern leopard frog are responsible for X-ray-elicited electroretinography (ERG) responses, as reported by Bachofer et al. (references [7]-[12]).

A main advantage of embodiments of the subject invention is that they can provide the same function/purpose as optogenetics while overcoming the light-associated limitations (in terms of diffusion-based resolution loss and attenuation-induced bound on depth) and being far less invasive. Identification of the X-ray sensitive protein (e.g., opsins in the retina of the Northern leopard frog) can lead to the creation of an X-opsin superfamily having members with a variety of X-ray-controllable functions, and this has far-reaching implications in a diverse set of fields including molecular neuroscience, behavioral neuroscience, and multimodality theranostics.

Optogenetics includes activation of specific neurons by stimulating genetically-modified neurons with visible light and opening opsin-coupled channels. Embodiments of the subject invention can overcome the limitations of optogenetics, discussed herein, by using X-rays for direct switching of specific neurons.

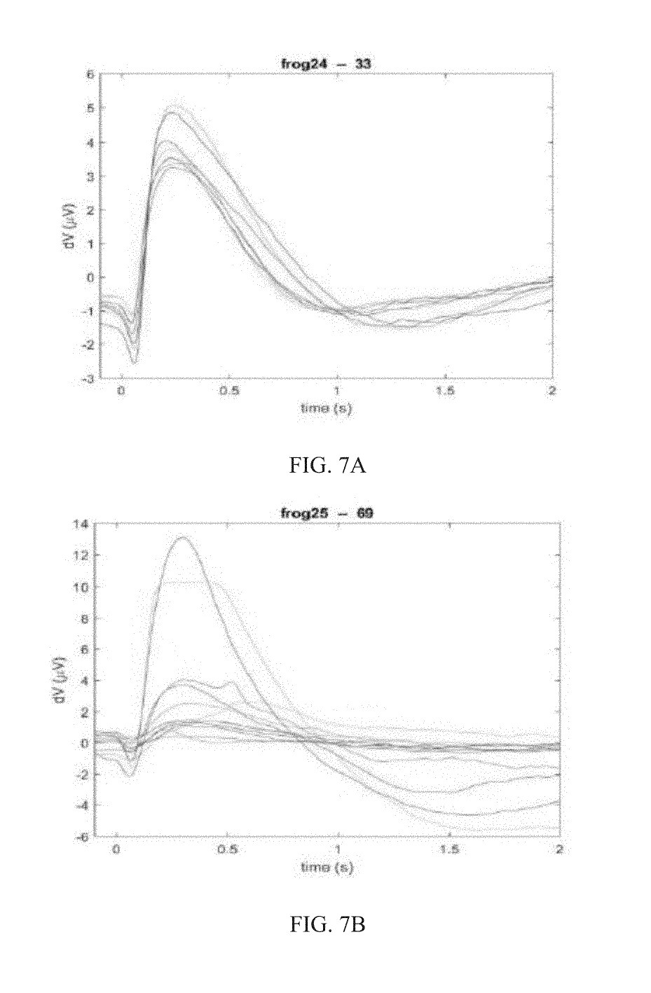

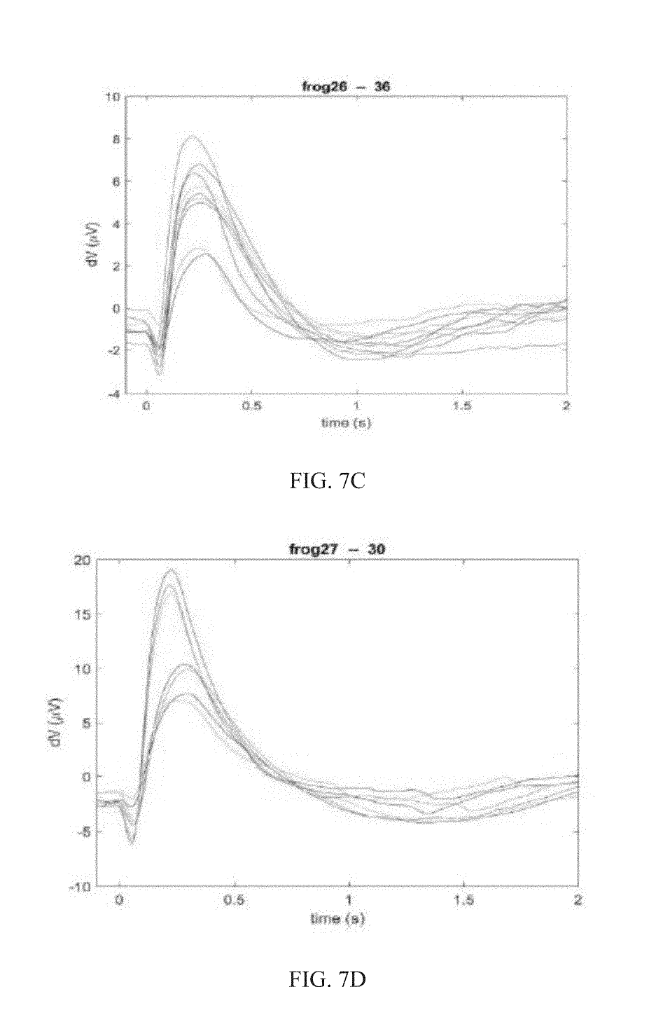

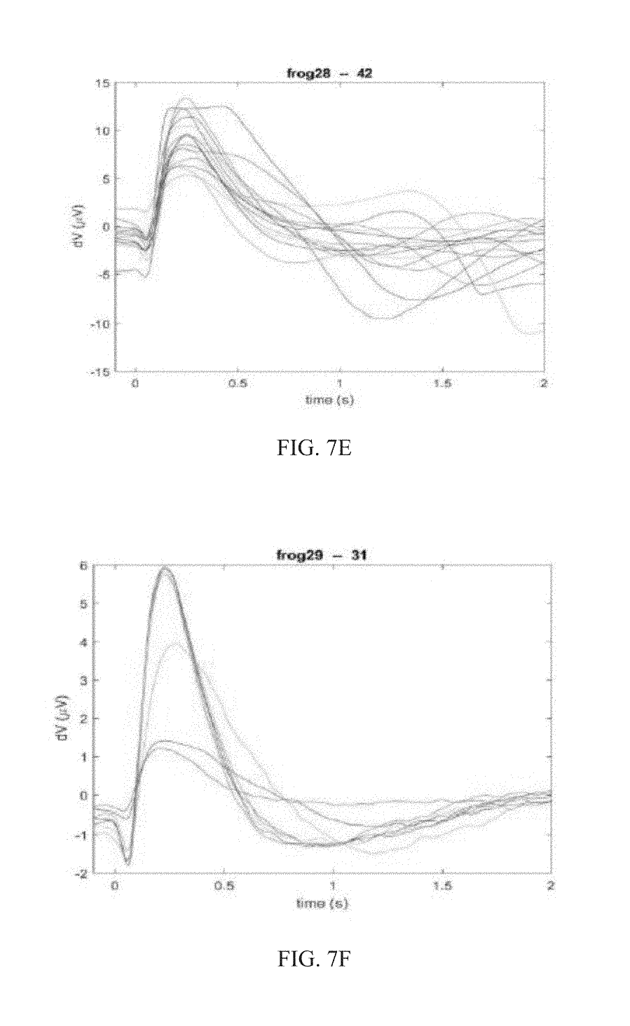

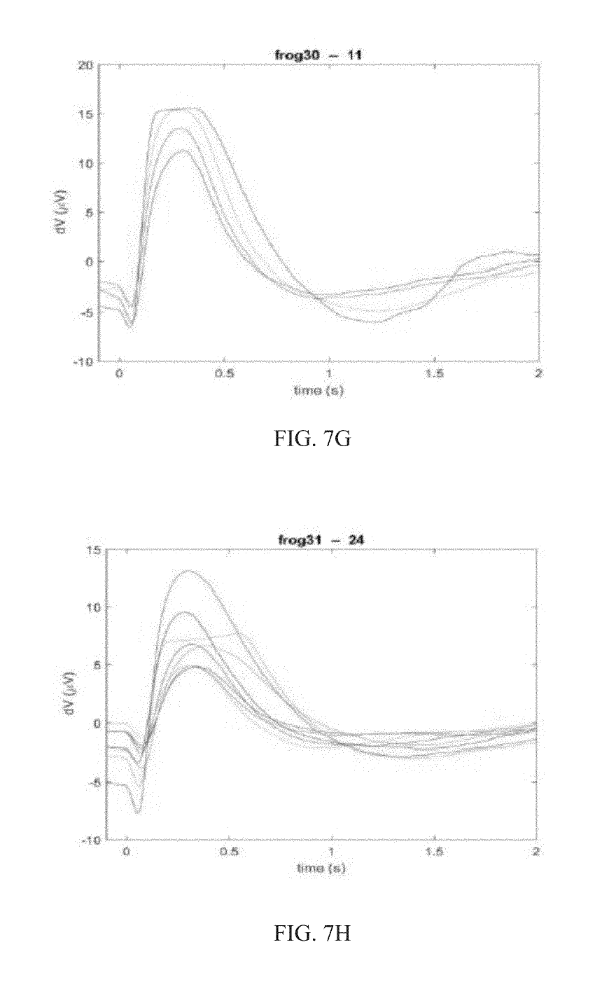

Electroretinogram (ERG), an electrophysiological response from light stimulation of the retina, results in a differential voltage potential. This can be measured by placing two conductive electrodes across the retina. Subsequently, light stimuli can be directed toward the retina, and the global changes in electrical potentials of the cells in the retina can be measured. In dark-adapted vertebrates, these changes occur after a flash stimulus that results in photoisomerizations of the retinal molecule in rod cells of a retina, indicated by a negative dip called an a-wave ([99]). Shortly after the rod-driven a-wave, the bipolar cells become activated and begin propagating action potentials to the ganglion cells. This bipolar cell activity is responsible for the large positive b-wave seen in visible ERGs as well as the first line of visual signal processing. FIGS. 7A-7H show examples of such light-evoked ERG waves. Stimuli that result in lower numbers of photoisomerizations lack the initial a-wave, though the b-wave remains.

As the front line of neuronal cells in the visual system, rods and cones convert incoming light stimuli to action potentials through their light-sensitive proteins, which, in the rod cells, are called rhodopsins. More generally, light sensing proteins are called opsins. These G-protein coupled receptors (GPCRs) bind covalently to the cofactor retinal, which is produced from Vitamin A and which is converted from 11-cis-retinal to all-trans-retinal in the presence of light. This conversion results in the conformational change in the opsin and activation of the associated G-protein and its second messenger cascade. This mechanism is analogous to the conversion of other stimuli (mechanical, chemical, or electrical) into cellular signals by neuronal cells throughout the vertebrate nervous system. Even the most basic eukaryotic cells have mechanisms by which light can be sensed, although rather than acting through activation of the coupled G-protein, these proteins are often channels/pumps that act to directly control the internal and external concentrations of ions and, therefore, the membrane potential of the cell. Depending on the ions to which these channels are privy, action potentials can either be elicited or quieted in the presence of light. Bacterial opsins (e.g., channelrhodopsin, bacteriorhodopsin, archaerhodopsin), along with designed chimeric light-sensitive channels and GPCRs, have been used for use in light-based therapies and basic research studies ([100]-[104]). This is referred to as optogenetics, and embodiments of the subject invention can perform similar cell behavioral control while being less invasive and penetrating tissues more deeply; no visible light is needed, only X-ray radiation/stimulation.

In many embodiments of the subject invention, the X-ray sensitive biomolecule used for controlling the behavior of one or more cells via X-ray stimulation is an opsin. For example, the X-ray sensitive biomolecule can be rhodopsin, such as rhodopsin from outer segments of rod cells in a retina (e.g., in a retina of a frog such as the Northern leopard frog). It is not completely clear if the X-ray sensitive protein interacts directly with the high energy photons to initiate a signal transduction cascade.

In certain embodiments, when providing stimulation to an organism, pharmacological intervention(s) can be used to knock out rhodopsin response to light or other aspects along the signal transduction cascade. In addition, random block and partial block designs could be employed to increase the power of observations and allow for parametric characterization of the phenomena observed, and/or high-precision servo motors in conjunction with optical methods can be used to ensure precise electrode placement minimizing human error. In some embodiments, 3D retina models that accommodate differential distribution and connections of cells can be used to help understand the effects of observed parameters on signal polarity, and/or oxygen perfusion can be used to ensure high blood oxygen levels in an organism.

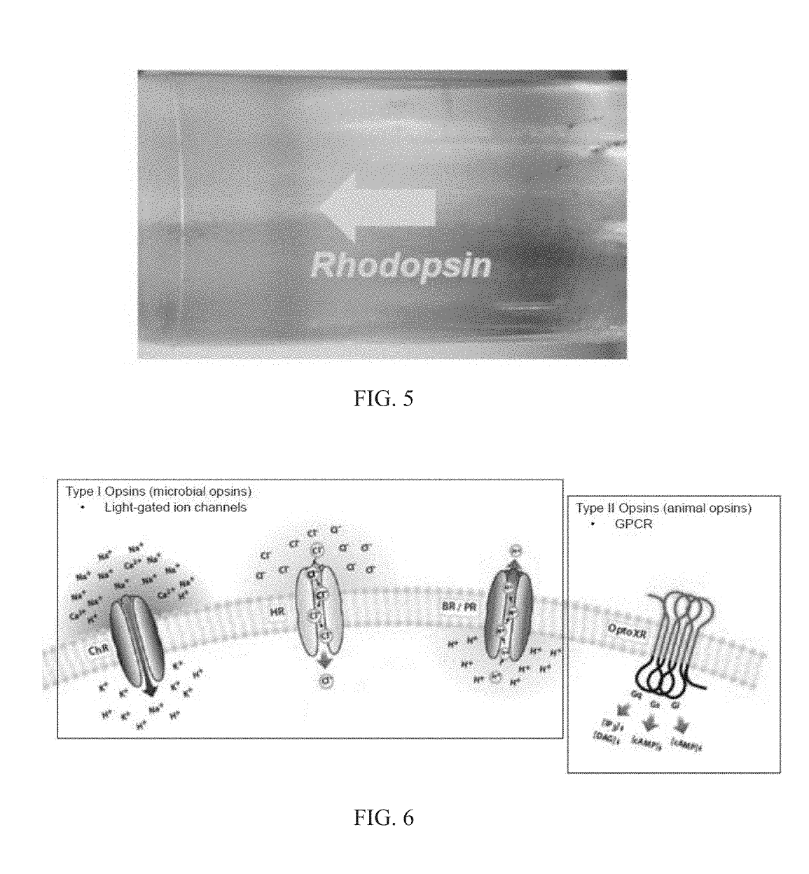

X-ray sensitive proteins other than those explicitly discussed herein may be identified, by for example using one of the methods depicted in FIG. 4. For example, a number of species of invertebrates such as the horseshoe crab and mantis shrimp have evolved to express retinal sensitivities well outside the visible spectrum and into the deep UV and IR spectra. It is therefore not impossible that other animal species have evolved with retinal proteins that are sensitive to X-ray. Rhodopsin can also be obtained from bacteria purified with a sucrose gradient, the result of which is shown in FIG. 5. In addition, while ERG can be used for an understanding of the retinal dynamics as it responds to various stimuli, patch-clamp technologies can also be employed to tease out cells and proteins that respond to the high-energy photons of an X-ray beam.

X-ray sensitive photoreceptors and phototransduction can be characterized using, for example, patch-clamp techniques. The mechanism underlying visible light phototransduction in vertebrate rod segments has been discussed in [139] and [140], and FIG. 24 shows a schematic view of the mechanism. Referring to FIG. 24, the rod outer segment (ROS) includes stacked internal membranes and disk membranes, enveloped by the plasma membrane. The main component of disks is rhodopsin (Rho). Phototransduction starts with the absorption of light, causes conformational changes of Rho*, and leads to formation of signaling Meta II. Meta II binds and activates photoreceptor-specific G protein molecules, transducins (Gt), by catalyzing the exchange of GTP for GDP on transducin's α subunit (Gtα) for its dissociation from the βγ dimer (Gtβγ). An activated transducin a subunit (Gtα*) activates cGMP-specific PDE to hydrolyze cGMP molecules to GMP, reducing [cGMP] and lowering the permeability of cGMP-gated cation channels in the rod plasma membrane. These events result in a hyperpolarization of the plasma membrane, generation of a neuronal signal at the synaptic terminal, changes in the rate of neurotransmitter release, and communication with other neurons.

Embodiments of the subject invention also provide image reconstruction methods using deep learning. A deep learning algorithm and/or deep neural network can be used to obtain a reconstructed image from raw data (e.g., features) obtained with medical imaging (e.g., CT, MM, X-ray). In a specific embodiment, a conventional (i.e., non-deep-learning) reconstruction algorithm can be used on the raw imaging data to obtain an initial image, and then a deep learning algorithm and/or deep neural network can be used on the initial image to obtain a reconstructed image. In many embodiments, a training set and/or set of final images can be provided to a deep network to train the network for the deep learning step (e.g., versions of what a plurality of final images should look like are provided first, before the actual image reconstruction, and the trained deep network can provide a more accurate final reconstructed image).

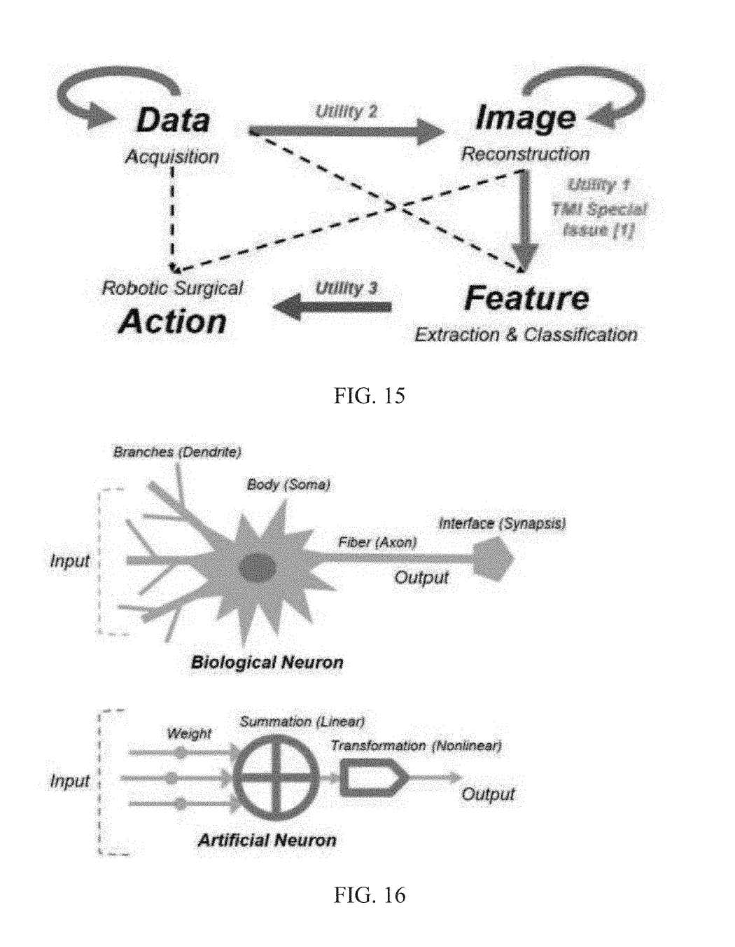

The combination of medical imaging, big data, deep learning, and high-performance computing promises to empower not only image analysis but also image reconstruction. It is well-known that there are two parts in the medical imaging field: (1) image formation/reconstruction, from data to images; and (2) image processing/analysis, from images to images (de-noising, etc.) or from images to features (recognition, etc.). FIG. 15 shows a schematic of deep imaging, a full fusion of medical imaging and deep learning.

As the center of the nervous system, the human brain contains many billions of neurons, each of which includes a body (soma), branching thin structures from the body (dendrites), and a nerve fiber (axon) reaching out. Each neuron is connected by interfaces (synapses) to thousands of neighbors, and signals are sent from axon to dendrite as electrical pulses (action potentials). Neuroscience views the brain as a biological computer whose architecture is a complicated biological neural network, where the human intelligence is embedded. In an engineering sense, the neuron is an electrical signal processing unit. Once a neuron is excited, voltages are maintained across membranes by ion pumps to generate ion concentration differences through ion channels in the membrane. If the voltage is sufficiently changed, an action potential is triggered to travel along the axon through a synaptic connection to another neuron. The dynamics of the whole neural network is far from being fully understood. Inspired by the biological neural network, artificial neurons can be used as elements of an artificial neural network. This elemental model linearly combines data at input ports like dendrites, and non-linearly transforms the weighted sum into the output port like the axon. FIG. 16 shows a schematic view of a biological neuron and an artificial neuron.

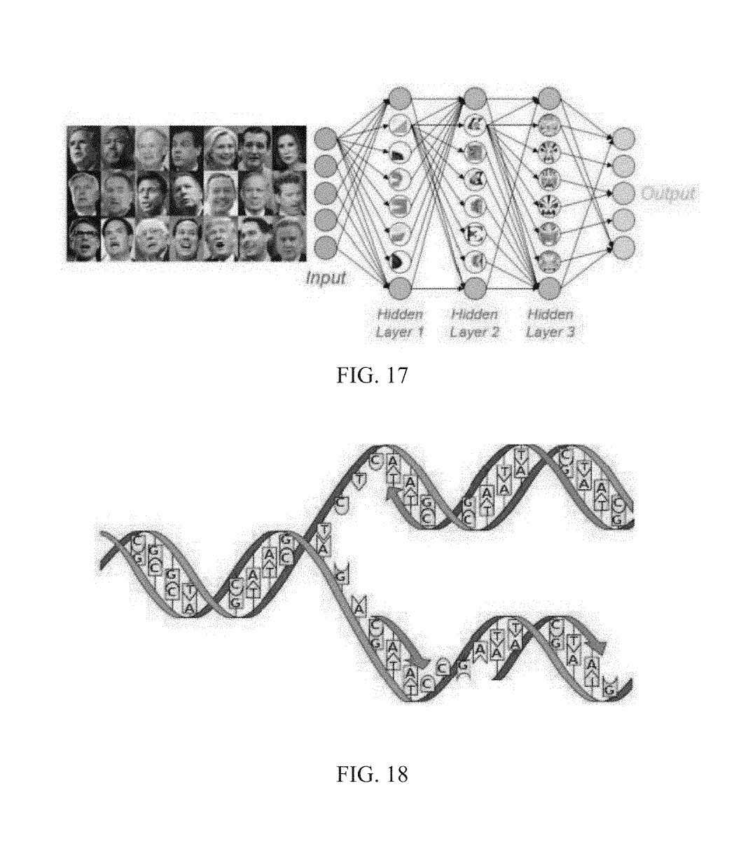

The major successes of deep networks are now well reported in computer vision, speech recognition, and language processing. Consider a neural network that works for face recognition. Referring to FIG. 17, there are many layers of neurons with inter-layer connections in a deep network. Data are fed into the input layer of the network, and weights associated with the neurons are typically obtained in a pre-training and fine-tuning process or a hybrid training process with a large set of unlabeled and labeled images. Results are obtained from the output layer of the network, and other layers are hidden from direct access. Each layer uses features from the previous one to form more advanced features. At earlier layers, more local features are analyzed such as edges, corners, and facial motifs. At later layers, more global features are synthesized to match face templates. Thanks to innovative algorithmic ingredients that have been developed, this deep learning mechanism has been made effective and efficient for feature extraction from images, and has demonstrated surprising capabilities. A deep network is fundamentally different from many other multi-resolution analysis schemes and optimization methods. A distinguished niche of deep networks is the nonlinear learning and optimization ability for nonconvex problems of huge dimensionality that used to challenge machine intelligence.

While FIG. 17 illustrates the process from images to features, it would be advantageous to go from projection/tomographic data to reconstructed images. The raw data collected for tomographic reconstruction can be considered as features of images, which are oftentimes approximated as linearly combined image voxel values, and more accurately modeled as nonlinear functions of the image parameters. Thus, image reconstruction is from raw data (features measured with tomographic scanning) to images, an inverse of the recognition workflow from images to features in FIG. 17. Embodiments of the subject invention can include image reconstruction from raw data to images using deep learning.

A classic mathematical finding of artificial neural networks is the so-called universal approximation theorem that, with a reasonable activation function, a feed-forward network containing only a single hidden layer may closely approximate an arbitrary continuous function on a compact subset when parameters are optimally specified ([74]). Then, the assumption on the activation function was greatly relaxed, leading to a statement that “it is not the specific choice of the activation function, but rather the multilayer feedforward architecture itself which gives neural networks the potential of being universal learning machines” ([75]). Although a single hidden layer neural network can approximate any function, it is highly inefficient to handle big data since the number of neurons would grow exponentially. With deep neural networks, depth and width can be combined to more efficiently represent functions to high precision, and also more powerfully perform multi-scale analysis, quite like wavelet analysis but in a nonlinear manner.

If the process from images to features is considered as a forward function, the counterpart from features to images can be thought of as an inverse function. Just like such a forward function has been successfully implemented in the deep network for many applications, so should be the inverse function for various tomographic modalities, both of which are guaranteed by the intrinsic potential of the deep network for a general functional representation, be it forward or inverse. Because the forward neural network is deep (many layers from an image to features), it is natural to expect that the inverse neural network should be also deep (many layers from raw data to an image). Despite special cases in which relatively shallow networks may work well, the neural network should be generally deep when the problem is complicated and of high dimensionality so that the aforementioned representation efficiency and multi-resolution analysis can be achieved through optimization of depth and width to combat the curse of dimensionality.

Consider computed tomography (CT) as a non-limiting example. It can be imagined that many CT reconstruction algorithms can be covered in the deep imaging framework. In the past, image reconstruction was focused on analytic reconstruction, and analytic reconstruction algorithms are present even in the intricate helical cone-beam geometry, which implicitly assume that data are accurate. With the increasing use of CT scans and associated public concerns on radiation safety, iterative reconstruction algorithms became gradually more popular. Many analytic and iterative algorithms should be able to be upgraded to deep imaging algorithms to deliver superior diagnostic performance.

When a projection dataset is complete, an analytic reconstruction would bring basically full information content from the projection domain to the image space even if data are noisy. If a dataset is truncated, distorted, or otherwise severely compromised (for example, limited angle, few-view, local reconstruction, metal artifact reduction, beam-hardening correction, scatter suppression, and motion restoration problems), a suitable iterative algorithm can be used to form an initial image. It is the image domain where a system of an embodiment of the subject invention can be good at de-noising, de-streaking, de-blurring, and interpretation. In other words, existing image reconstruction algorithms can be utilized to generate initial images, and then deep networks can be used to do more intelligent work based on initial images. This two-stage approach is advantageous as an initial strategy for three reasons. First, all the well-established tomographic algorithms are still utilized. Second, the popular deep networks with images as inputs can be easily transferred. Third, domain-specific big data can be fully incorporated as unprecedented prior knowledge. With this approach, the neural network is naturally deep because medical image processing and analysis can be effectively performed by a deep network. Similarly, a sinogram can be viewed as an image, and a deep learning algorithm can be used to improve a low-dose or otherwise compromised sinogram. This transform from a poor sinogram to an improved sinogram is another type of image processing task, and can be performed via deep learning. Then, a better image can be reconstructed from the improved sinogram.

As mathematically discussed above in terms of forward and inverse functions, both analytic and iterative reconstruction algorithms can be implemented or approximated with deep networks. This viewpoint can also be argued from an algorithmic perspective. Indeed, either the filtered back-projection (FBP) or simultaneous algebraic reconstruction technique (SART) can be easily formulated in the form of parallel layered structures (for iterative reconstruction, the larger the number of iterations, the deeper the network will be). Then, a straightforward method for deep imaging, according to an embodiment, can be just from raw data to an initial image through a neural network modeled after a traditional reconstruction scheme, and then from the initial image to a final image through a refinement deep network. This streamlined procedure can be extended to unify raw data pre-processing, image reconstruction, image processing, and image analysis, leading to even deeper network solutions. In the cases of missing or distorted data, the deep network can make a best link from measured data to reconstructed images in the sense of the best nonlinear fit in terms of big data.

The above considerations apply to other medical imaging modalities because all these biomedical imaging problems are associated with similar formulations in the general category of inverse problems. To the first order approximation, a majority of medical imaging algorithms have Fourier or wavelet transform related versions, and could be helped by some common deep networks. For nonlinear imaging models, deep imaging should be an even better strategy, given the nonlinear nature of deep networks. While the multimodality imaging trend promotes a system-level integration, deep imaging might be a unified information theoretic framework or a meta-solution to support either individual or hybrid scanners.

The imaging algorithmic unification is consistent with the successes in the artificial intelligence field in which deep learning procedures follow very similar steps despite the problems appearing rather different, such as chess playing, electronic gaming, face identification, and speech recognition. Just as a unified theory is preferred in the physical sciences, a unified medical imaging methodology would have advantages so that important computational elements for network training and other tasks could be shared by all the modalities, and the utilization of inter-modality synergy could be facilitated since all the computational flows are in the same hierarchy consisting of building blocks that are artificial neurons and also hopefully standard artificial neural circuits.

A key prerequisite for deep imaging is a training set that spans the space of all relevant cases. Otherwise, even an optimized deep network topology could be disappointing in real world applications. Also, it remains an open issue which reconstruction schemes would be better—classic analytic or iterative algorithms, deep networks, hybrid configurations, or unified frameworks. The answer can be application-dependent. For a clean dataset, the conventional method works well. For a challenging dataset, the deep network can be used. In any case, deep learning can be (theoretically and/or practically) relevant to medical imaging.

From a perspective of theoretical physics, the concept of the renormalization group (RG, related to conformal invariance by which a system behaves the same way at different scales) has been utilized for understanding the performance of deep learning. Deep learning may be an RG-like scheme to learn features from data. Each neuron is governed by an activation function which takes data in the form of an inner product, instead of input data directly. The inner product is computed as a sum of many products of paired data, which can be visualized as a double helix as shown in FIG. 18, in which the paired results between the double helix are lumped together. In other words, it is suggested that the inner product is the fundamental construct for deep learning, and in this sense it serves as “DNA” for data analysis. This view is mathematically meaningful because most mathematical transforms including matrix multiplications are calculated via inner products. The inner products are nothing but projections onto appropriate bases of the involved space. Cross- and auto-correlations are inner products, common for feature detection and filtration. Projections and back-projections are inner products as well. Certainly, the inner product operation is linear, and methods should not be limited to linear spaces. Then, the nonlinear trick comes as an activation function (see also FIG. 16).

In a deep network, the alternating linear and nonlinear processing steps seem to hint that the simplest linear computational elements (inner products) and simplest nonlinear computational elements (monotonic activation functions) can be organized to perform highly complicated computational tasks. Hence, the principle of simplicity applies not only to physical sciences but also to information/intelligence sciences, and the multi-resolution phenomena seems merely a reflection of this principle. When inner products are performed, linear elements of machine intelligence are realized; when the activation steps (in a general sense, other effects are included such as pooling and dropout) are followed, the non-linear nature of the problem is addressed; so on and so forth, from bottom up (feed forward) and from top down (back propagation).

Most existing analytic and iterative algorithms were designed for linear imaging problems. If the linear system model is accurate, at the first look, there appears no need to trade analytic and statistical insight for nonlinear processing advantages of deep networks through intensive tedious training. Nevertheless, even in that case, deep imaging is conceptually simple, universally applicable, and the best platform to fully utilize domain specific knowledge extracted from big data. Such comprehensive contextual prior knowledge cannot be utilized by iterative likelihood/Bayesian algorithms, which are nonlinear but limited to compensation for statistical fluctuation. Additionally, with the principle of simplicity, deep imaging is preferred, using the analogy of digital over analog computers.

Deep learning has achieved impressive successes in practice but a decent theory remains missing. Open issues include why ConvNet works well, how many layers, neurons, and free parameters are needed, and questions about local minima, structured predictions, short-term/working/episodic memories, and better learning methods. Also, slightly different images could be put into distinct classes, and random images could be accepted into a class with a high confidence level.

In medical tomography, image reconstruction is generally not unique from a finite number of projections, but the influence of non-uniqueness is avoided in practice where priori knowledge is present that an underlying image is band-limited, and a set of sufficiently many data in reference to the bandwidth can be collected. In the area of compressed sensing, while this technique produces visually pleasing images, tumor-like features may sometimes be hidden or lost ([83]). Nevertheless, these features were constructed based on the known imaging geometry and the algorithm, which would not likely be encountered in clinical settings. Most theoretical analyses on compressed sensing methods state the validity of the results with the modifier “with an overwhelming probability”, such as in [84]. Hence, flaws of deep learning should be very fixable in the same way or insignificant in most cases, because it can be imagined that if the types of training data are sufficiently representative and the structure of a deep network is optimized, prior knowledge (including but not limited to statistical likelihood) can be fully presented for superior image reconstruction.

More aggressively speaking, deep imaging could outperform conventional imaging with statistical, sparsity, and low rank priors, because information processing is nonlinear with a deep network, global through a deeply layered structure, and the best bet with the detailed prior knowledge learned from big data. This is in sharp contrast to many traditional regularizers that are linear, local, or ad hoc. Although the state of the art results obtained with over-complete wavelet frames or dictionary atoms bear similarities to that with auto-encoders, the wavelet and dictionary based features are both linear and local, and should be theoretically inferior to nonlinear and global representations enabled by a deep network.

Of particular relevance to deep imaging is unsupervised and supervised training of a deep network with big data, or the relationship between big data and deep learning for medical imaging. In the clinical world, there are enormous image volumes but only a limited amount of them were labeled, and patient privacy has been a hurdle for medical imaging research. Nevertheless, the key conditions are becoming ready for big data and deep learning to have an impact on medical imaging research, development, and application. First, big data are gradually accessible to researchers. For example, in the National Lung Screening Trial (NLST) project ([85]), over 25,000 patients went through three low-dose CT screenings (T0, T1, and T2) at 1-year intervals, which resulted in more than 75,000 total datasets. Second, deep learning can be implemented via a pre-training step without supervision or a hybrid training process so that intrinsic image features are learned to have favorable initial weights, and then performs backpropagation for fine-tuning. Third, hardware for big data, deep learning, and cloud computing is commercially available and being rapidly improved. Therefore, deep learning can be transferred to medical image reconstruction.

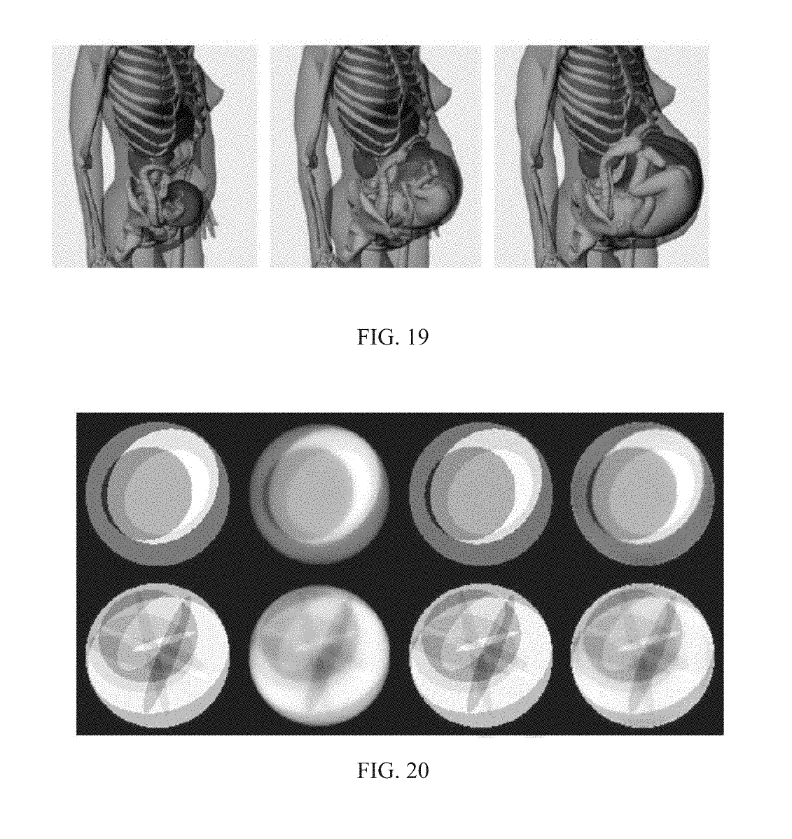

Because of the visible human project ([86]) and other similar efforts, realistic image volumes of the human bodies in different contrasts (e.g., CT and MRI) are readily available. With deformable matching methods, many realistically deformed image volumes can be produced. Also, physiological and pathological features and processes can be numerically added into an image volume or model ([87]); see also FIG. 19. Such a synthetic big data could be sufficient for deep imaging.

Supposing that a deep network is well trained, its structure should be stable through re-training with images obtained through locally and finely transformed previously-used images. In other words, moderate perturbation can be an easy mechanism to generate big data. Additionally, this invariance may help characterize the generic architecture of a deep imager.

A deep neural network, and artificial intelligence in general, can be further improved by mimicking neuroplasticity, which is the ability of the brain to grow and reorganize for adaption, learning, and compensation. Currently, the number of layers and the number of neurons per layer in a deep network are obtained using the trial and error approach, and not governed by any theory. In reference to the brain growth and reorganization, the future deep network could work in the same way and become more adaptive and more powerful for medical imaging. As time goes by, it may be possible to design deep networks that are time-varying, reconfigurable, or even have quantum computing behaviors ([87]).

Deep learning is not only a new wave of research, development, and application in the field of medical imaging but also a paradigm shift. From big data with deep learning, unprecedented domain knowledge can be extracted and utilized in an intelligent framework from raw data to final image until clinical intervention. This can be empowered with accurate and robust capabilities to achieve optimal results cost-effectively, even for data that are huge and compromised, as well as for problems that are nonlinear, nonconvex, and overly complicated.

A greater understanding of the embodiments of the present invention and of their many advantages may be had from the following examples, given by way of illustration. The following examples are illustrative of some of the methods, applications, embodiments, and variants of the present invention. They are, of course, not to be considered as limiting the invention. Numerous changes and modifications can be made with respect to the invention.

### Example 1

A visible/X-ray ERG prototype was applied to elicit and measure retinal responses of the Norther leopard frog (R. pipiens) from visible and X-ray stimuli. Eight Northern leopard frogs were used once, unless otherwise noted, in this round of signal acquisition with the ERG system. Each frog was dark-adapted overnight and handled in a room illuminated with a low intensity, 650 nm high pass filtered light (Roscolux #27, Rosco Laboratories). The frogs were anesthetized in the dark room by immersion in 1 g/L solution of MS-222 (pH buffered to −7 with NaHCO3) for 2-8 minutes. After pulsing, each animal was revived in dH₂O until fully recovered. During the ERG acquisitions, the frogs did not receive supplemental oxygen. Table 1 shows each of the visible, X-ray, and solenoid-only ERG recordings of the frogs. The cumulative amount of radiation prior to the visible ERG recording is also listed.

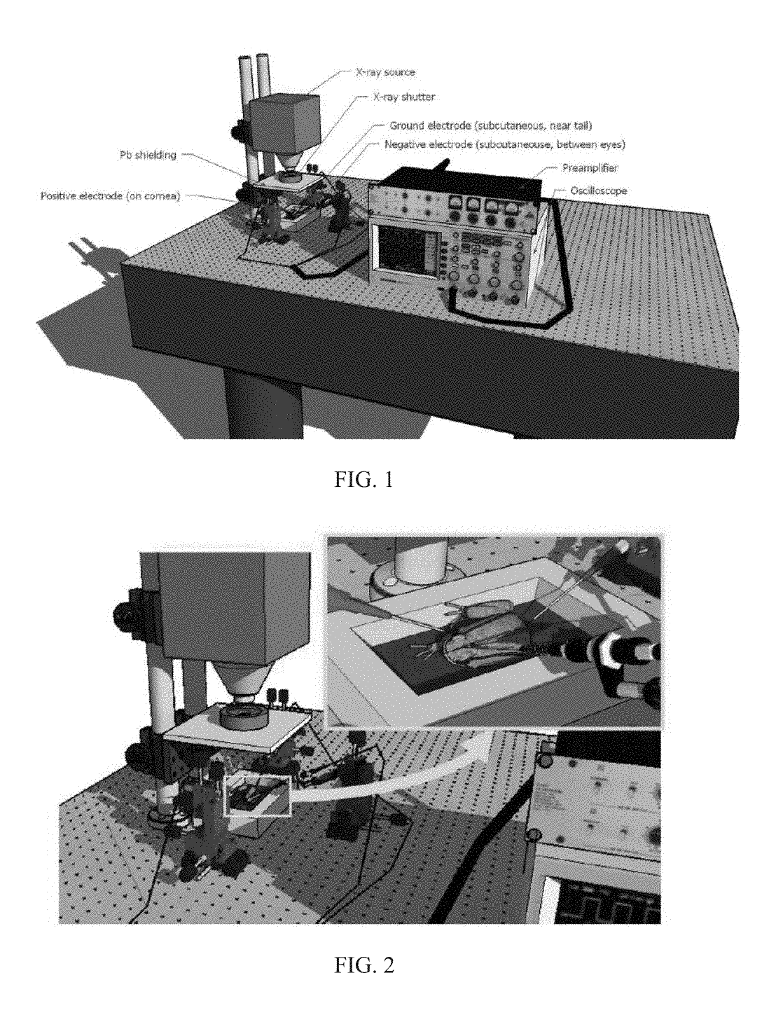

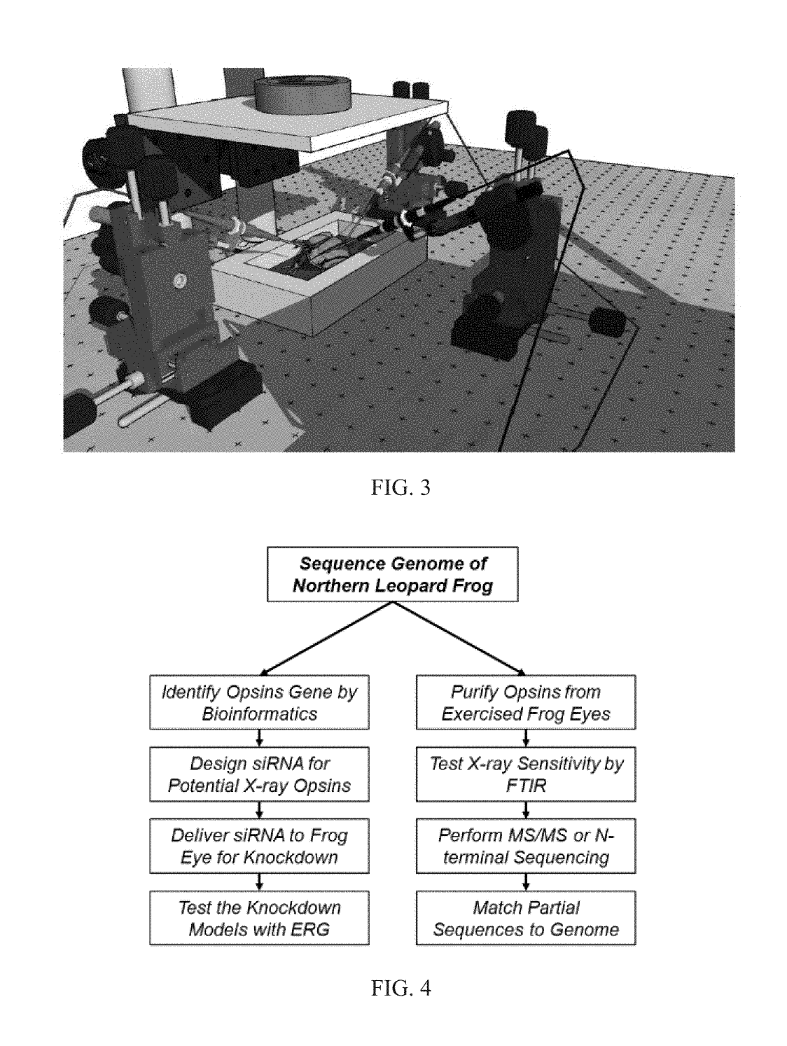

GRASS subdermal, platinum needle electrodes (NATUS) were used for the ERG signal acquisitions. Held in place by an adjustable arm, the (+) electrode was laid across the frog's cornea and its connection was sealed with Goniovisc solution; the (−) and GND electrodes were placed subcutaneously between the eyes and in the tail, respectively. Each frog was also subject to a number of control measurements in which the electrodes were place subcutaneously across the back. The electrode placement was moved and assessed until a steady-state differential potential was found, void of heart-beat or other artifacts. If signal-to-noise decreased over the time of the signal acquisition, the electrodes were adjusted. Electrode placements and the system setup can be seen in FIGS. 1-3.

Both visible and x-ray ERG signals were acquired in the LabChart (ADInstruments) via the differential amplifier and headstage (A-M Systems) and PowerLab 2/26 (ADInstruments). The stimuli were coordinated in the LabChart software so that recording started 5 seconds prior to the stimulus pulse and continued for 8 seconds after the pulse. The head stage amplified the signal 10×, and the amplifier gain was set to 10,000×. The amplifier filters were set to low-pass 0.1 kHz and high-pass 1 Hz. Additionally, a 60 Hz digital low-pass filter was applied in the LabChart/MATLAB software to clean-up the high frequency noise components of the raw ERG signals.

The visible light pulses were administered from a 470 nm, 26.5 mW mounted LED (Thorlabs, Inc.), which was driven by the T-cube LED driver (Thorlabs, Inc.). The light pulses were software controlled using LabChart (ADInstruments) via the analog output channels from the PowerLab (ADInstruments). The light was placed about 2.5-4 cm from the eye of the frog. All visible stimulation was done with 20 msec pulse width, and the intensity of the flashes were measured to be 232 μW at the position of the frogs' eyes.

A Hamamatsu microfocus X-ray source (L10101, 20-100 kVp, 50-200 μA) was used to produce the X-ray stimuli administered to the frogs. All stimulation was done at 100 kVp and 75 μA. A lead shutter, mounted on a solenoid and wired to a MOSFET transistor, was used to block and create X-ray pulses. The solenoid was controlled using LabChart (ADInstruments) via the analog output channels from the PowerLab (ADInstruments). The minimum pulse width that the solenoid was capable of producing was 200 msec. Pulses ranging from 200-1000 msec were administered to the frogs, which were placed about 20 cm from the X-ray tube and aligned with the X-ray beam. Various lag times were tested between pulses. Visible responses were also interweaved between various X-ray exposures as controls and comparisons to before exposure measurements.

Dose estimations were calculated using the manufacturer provided dose rate for the microfocus source, 76 R/min, measured 30 cm from the focal spot while the source was being operated at 100 kVp and 200 μA. Pulse dose estimations were calculated with the following relationship conversions:

$$Estimated dose = ( 76 \frac{R}{min} ) ( \frac{1000 \frac{mR}{R}}{60 \frac{sec}{min}} ) ( \frac{{V}^{2} * A * {( 30 cm )}^{2}}{{( 100 kVp )}^{2} * ( 300 µA ) * {d}^{2}} ) = \frac{213.75 mR}{200 msec pulse} \qquad ( Eq . 1 )$$

where d is the measured distance from the source to the frog, V is the operating tube voltage, and A is the operating tube current. This dose rate does not take into account the beam collimation; however, assuming utilization of only 1% of the X-ray dose, the 2 mR (milli-Rads) per 200 msec pulse that results is in the ballpark for the known threshold for X-ray induced ERG.

The recordings were trimmed to encompass the 5 seconds on either side of the pulse. In order to eliminate recordings that were dominated by noise rather than by the light impulse responses, the primary peaks were compared to the pre-impulse signal behavior, quantified by signal range, derivative, and magnitude relative to rest of the measurements made for that frog. Pulses that were determined to have no response were analyzed as noise and fit to a single line.

TABLE 1
Mean exposure as well as time of occurrence and magnitude of the a- and b-waves are shown separated
by the amount of X-ray exposure prior to each visible response. Three levels of exposure were initially determined -
low, moderate, and high, which correspond to 10⁻⁶, 10⁻⁵, and 10⁻⁴ mGy (milli-Gray) of exposure,
respectively. The last row is a combination of all of the exposure levels.

| Response | | | | | Recovery | | | | |
|---|---|---|---|---|---|---|---|---|---|
| CC | DD | EE | FF | R² | GG | HH | II | JJ | R² |

Frog 24

| | | | | | | | | | | |
|---|---|---|---|---|---|---|---|---|---|---|
| Visible - | −2029.9 ± | 721.5 ± | −37.0 ± | −1.0 ± | 0.98 ± | 7.2 ± | −10.7 ± | −2.6 ± | 5.1 ± | 0.99 ± |
| eye | 773.8 | 267.1 | 19.2 | 0.3 | .007 | 2.6 | 4.3 | 2.8 | 1.0 | 0.004 |
| (n = 8) | | | | | | | | | | |

Frog 25

| | | | | | | | | | | |
|---|---|---|---|---|---|---|---|---|---|---|
| Visible - | −833.0 ± | 367.9 ± | −26.8 ± | −0.11 ± | 0.99 ± | 2.5 ± | −3.1 ± | −4.5 ± | 5.3 ± | 0.98 ± |
| eye | 1334.0 | 518.1 | 35.7 | 0.74 | 0.007 | 4.3 | 13.4 | 14.4 | 6.5 | 0.01 |
| (n = 13) | | | | | | | | | | |

Frog 26

| | | | | | | | | | | |
|---|---|---|---|---|---|---|---|---|---|---|
| Visible - | −2407.4 ± | 908.7 ± | −53.0 ± | −1.0 ± | 0.98 ± | 1.2 ± | 7.9 ± | −20.9 ± | 10.0 ± | 0.99 ± |
| eye | 973.1 | 313.2 | 15.9 | 0.4 | 0.005 | 8.8 | 20.0 | 13.0 | 2.7 | 0.003 |
| (n = 9) | | | | | | | | | | |
| XP - eye | −1161.4 ± | 314.3 ± | 37.4 ± | −2.5 ± | 1.00 ± | 20.9 ± | −27.4 ± | −6.0 ± | 11.1 ± | 0.99 ± |
| (n = 3) | 211.2 | 50.2 | 17.8 | 1.8 | 0.0001 | 28.0 | 44.1 | 17.7 | 2.6 | 0.01 |
| XN - eye | 346.6 ± | −103.1 ± | −4.5 ± | −0.18 ± | 1.00 ± | −5.19 ± | 1.9 ± | 7.7 ± | −4.3 ± | 0.99 ± |
| (n = 3) | 119.8 | 25.7 | 1.5 | 0.33 | 0.001 | 8.8 | 23.3 | 14.9 | 1.9 | 0.009 |
| XN - back | 6.7 | −8.6 | 6.3 | −0.24 | 0.99 | 0.46 | −0.41 | −1.5 | 1.3 | 0.95 |
| (n = 1) | | | | | | | | | | |

Frog 27

| | | | | | | | | | | |
|---|---|---|---|---|---|---|---|---|---|---|
| Visible - | −5382.5 ± | 2049.3 ± | −123.5 ± | −2.3 ± | 0.99 ± | −8.8 ± | 42.4 ± | −61.4 ± | 25.3 ± | 0.99 ± |
| eye | 3587.8 | 1210.8 | 69.2 | 0.5 | 0.002 | 13.8 | 34.7 | 30.5 | 9.1 | 0.006 |
| (n = 7) | | | | | | | | | | |
| XP - eye | −283.6 ± | 100.3 ± | 2.6 ± | −0.15 ± | 1.00 ± | 11.3 ± | −5.9 ± | −7.1 ± | 4.1 ± | 0.97 ± |
| (n = 4) | 65.0 | 28.0 | 5.0 | 0.22 | 0.003 | 35.2 | 50.2 | 22.2 | 3.0 | 0.02 |

Frog 28

| | | | | | | | | | | |
|---|---|---|---|---|---|---|---|---|---|---|
| Visible - | −3101.1 ± | 1155.9 ± | −55.9 ± | −1.1 ± | 0.99 ± | 10.1 ± | −7.5 ± | −18.8 ± | 14.3 ± | 0.98 ± |
| eye | 1206.0 | 465.6 | 42.0 | 1.3 | 0.009 | 17.3 | 37.5 | 26.6 | 5.6 | 0.017 |
| (n = 16) | | | | | | | | | | |
| XP - eye | −124.3 ± | 35.2 ± | 3.8 ± | −0.14 ± | 1.00 ± | 2.5 ± | −1.4 ± | −2.6 ± | 1.7 ± | 0.98 ± |
| (n = 3) | 64.2 | 21.0 | 0.6 | 0.21 | 0.0007 | 1.3 | 4.9 | 3.3 | 0.7 | 0.01 |
| XN - back | −116.0 ± | 50.5 ± | −67.7 ± | −1.5 ± | 1.00 ± | −31.7 ± | 72.6 ± | −26.0 ± | −12.3 ± | 0.99 ± |
| (n = 2) | 50.6 | 15.0 | 8.8 | 1.6 | 8.5e−5 | 9.9 | 21.0 | 11.4 | 3.2 | 0.003 |

Frog 29

| | | | | | | | | | | |
|---|---|---|---|---|---|---|---|---|---|---|
| Visible - | −1876.1 ± | 700.9 ± | −41.3 ± | −0.67 ± | 0.99 ± | −1.9 ± | 13.2 ± | −20.5 ± | 8.2 ± | 0.99 ± |
| eye | 981.9 | 375.9 | 25.2 | 0.26 | 0.001 | 3.9 | 13.9 | 15.2 | 4.8 | 0.006 |
| (n = 9) | | | | | | | | | | |

Frog 30

| | | | | | | | | | | |
|---|---|---|---|---|---|---|---|---|---|---|
| Visible - | −2679.3 ± | 1147.7 ± | −44.6 ± | −3.5 ± | 0.97 ± | 5.1 ± | 16.9 ± | −55.3 ± | 29.1 ± | 0.98 ± |
| eye | 793.0 | 349.7 | 47.0 | 1.0 | 0.02 | 13.1 | 34.1 | 22.9 | 2.2 | 0.006 |
| (n = 4) | | | | | | | | | | |

Frog 31

| | | | | | | | | | | |
|---|---|---|---|---|---|---|---|---|---|---|
| Visible - | −1350.7 ± | 638.1 ± | −39.3 ± | −2.0 ± | 0.98 ± | 3.2 ± | 4.7 ± | −24.7 ± | 15.4 ± | 0.98 ± |
| eye | 957.6 | 406.3 | 33.8 | 1.6 | 0.03 | 7.0 | 17.0 | 15.9 | 6.8 | 0.004 |
| (n = 8) | | | | | | | | | | |
| XP - eye | −109.6 ± | 37.7 ± | 5.1 ± | −0.46 ± | 1.00 ± | 2.3 ± | −1.1 ± | −3.4 ± | 2.1 ± | 0.99 ± |
| (n = 4) | 52.0 | 16.7 | 0.7 | 0.13 | 0.0005 | 4.3 | 6.5 | 2.9 | 0.6 | 0.006 |
| XN - back | 152.9 ± | −61.9 ± | −0.16 ± | −0.32 ± | 0.99 ± | 0.89 ± | −4.4 ± | 6.7 ± | −3.0 ± | 9.7 ± |
| (n = 5) | 109.8 | 44.2 | 3.2 | 0.22 | 0.01 | 1.7 | 4.0 | 2.7 | 0.5 | 0.01 |

The peaks of the a- and b-waves were identified and used for quantification of the full-width at half min/max (FWHM) and area under the curve (AUC) features. In addition to these, peak time and peak magnitude were used in the statistical analyses of the response behaviors.

Due to the main focus being hardware optimization and model exploration, the statistical power of the data to elucidate significant differences between data features, piecewise polynomial coefficients, and fit statistics with parametric methods was not as high as it could have been (<50%—calculated post-hoc for univariate paired t-tests using G*Power). Therefore, nonparametric bootstrap methods utilizing the R library sm were used to compare density distributions of ERG responses based on factors such as response type (visible, X-ray positive, X-ray negative, and noise) and pre-exposure (yes or no). The whole feature-set was compared in a univariate fashion, and p-values were corrected for multiple comparisons.

The analyzed response data was separated into four types of signals. The “visible light” ERG was characterized by the well-documented features of the a- and b-waves. Further, in many of the recorded responses, the oscillatory potentials at the peak of the b-wave were also witnessed. FIGS. 7A-7H show a summary of all the “visible” ERGs.

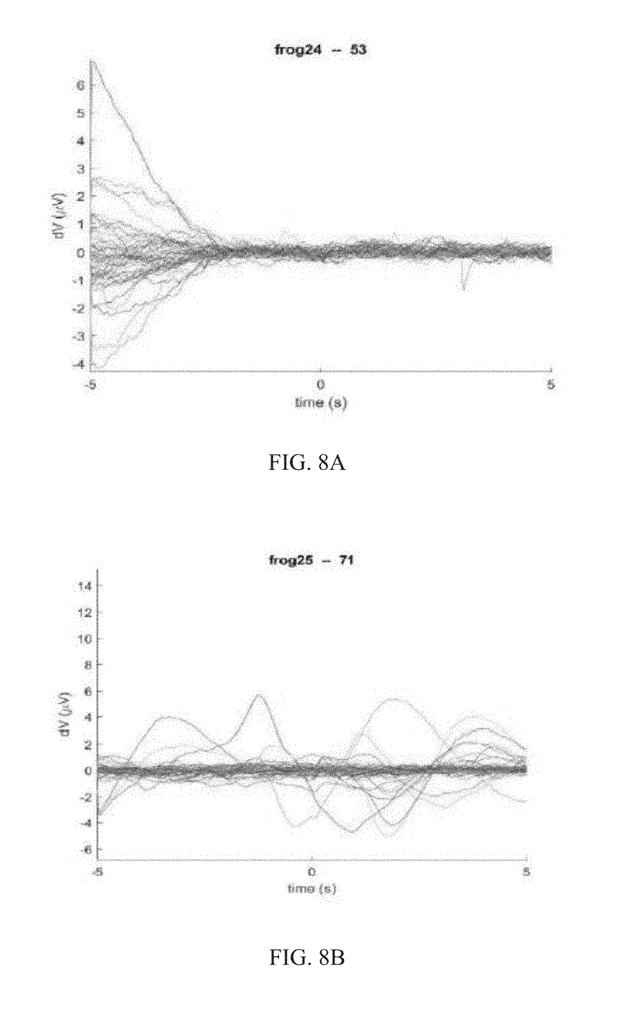

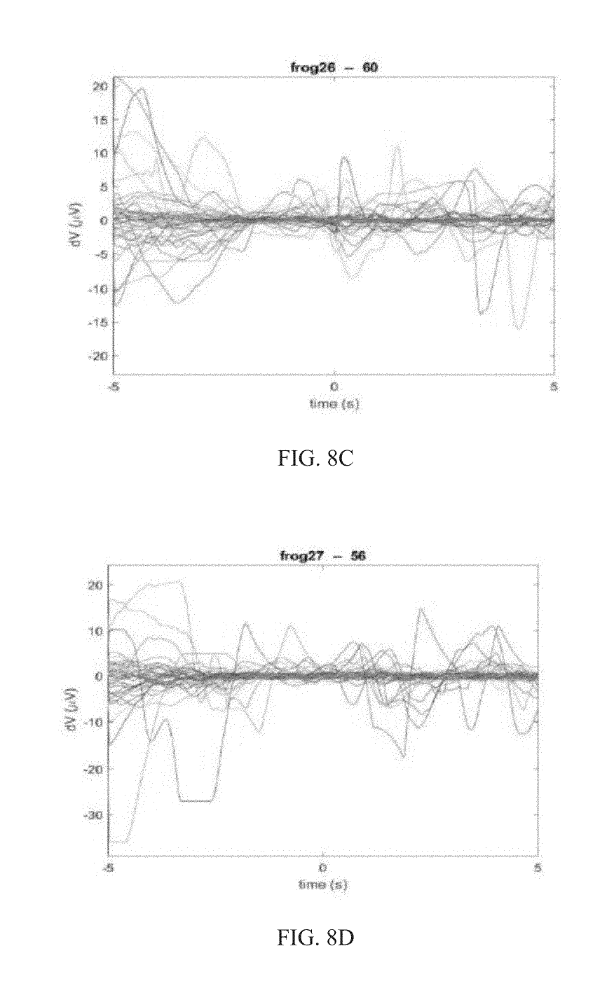

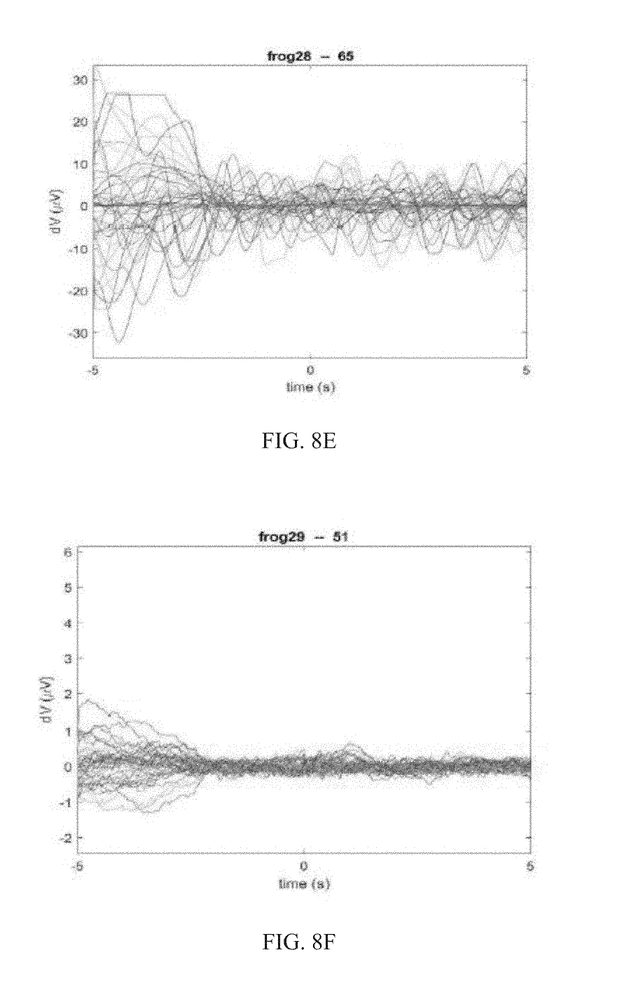

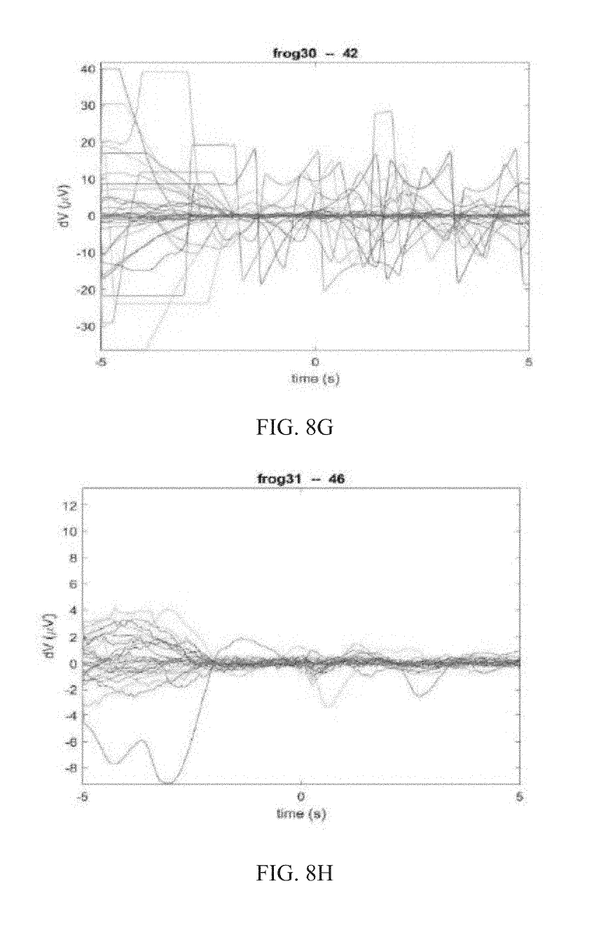

The “noise” or “no response” ERG was seen in the majority of the recordings during X-ray stimulation as well as a few of the visible stimuli recordings. These are all similar in that no measurable (relative to a threshold related to signal deviation) peaks were located in the signal; however, there were disparities in how noisy the signals in this group were. Additionally, some clear signals were deemed “no response” and other clear non-signals were deemed a measurable response. The line between response and none was blurry at times. FIGS. 8A-8H show a summary of all the “noise” ERGs.

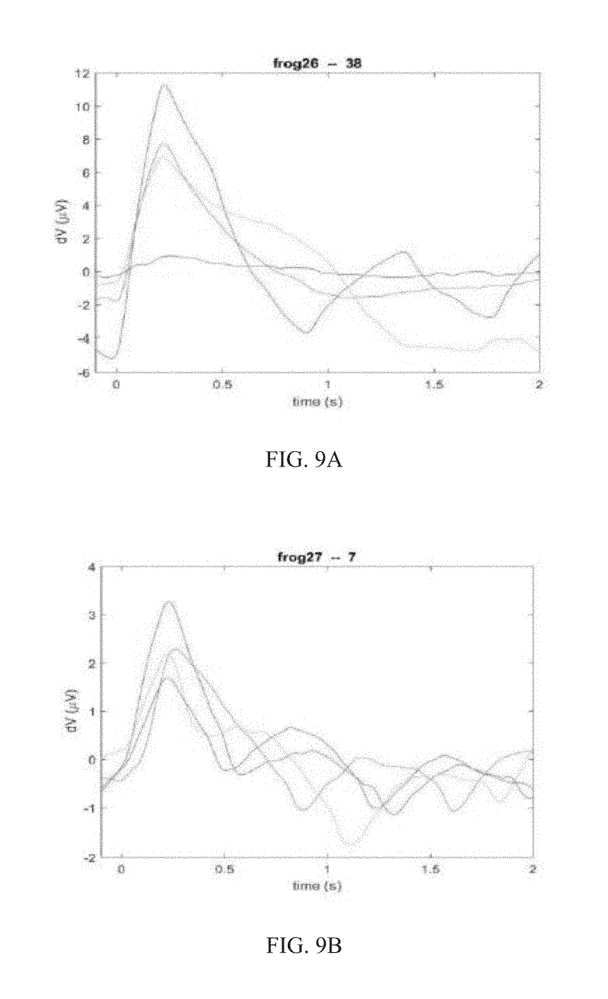

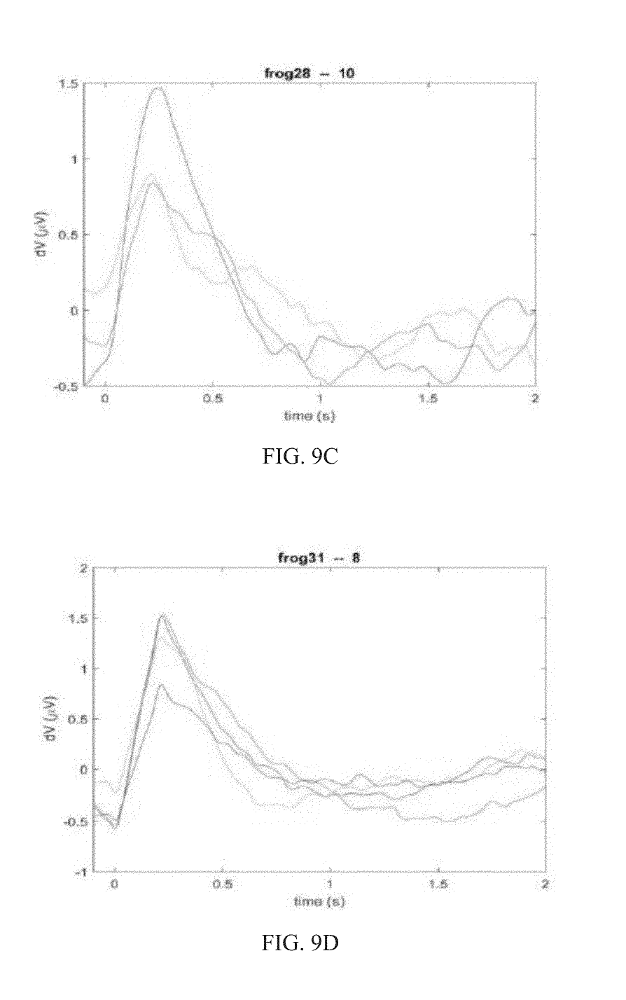

The “positive only” ERG was seen in four frogs, exclusively after short (200 msec—shortest possible with the solenoid-driven aperture) X-ray pulses. These signals are monotonic from the pulse at t=0 to the peak. Notably, these peaks resembled the signals reported previously ([7]-[12]) as well as visible pulses. FIGS. 9A-9D show a summary of the “positive only” x-ray ERG responses.

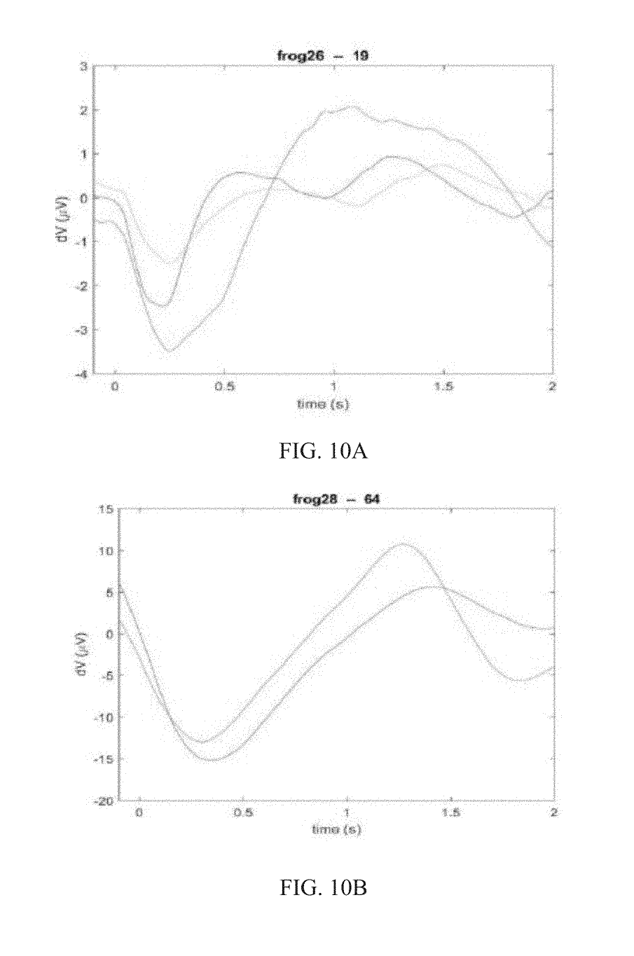

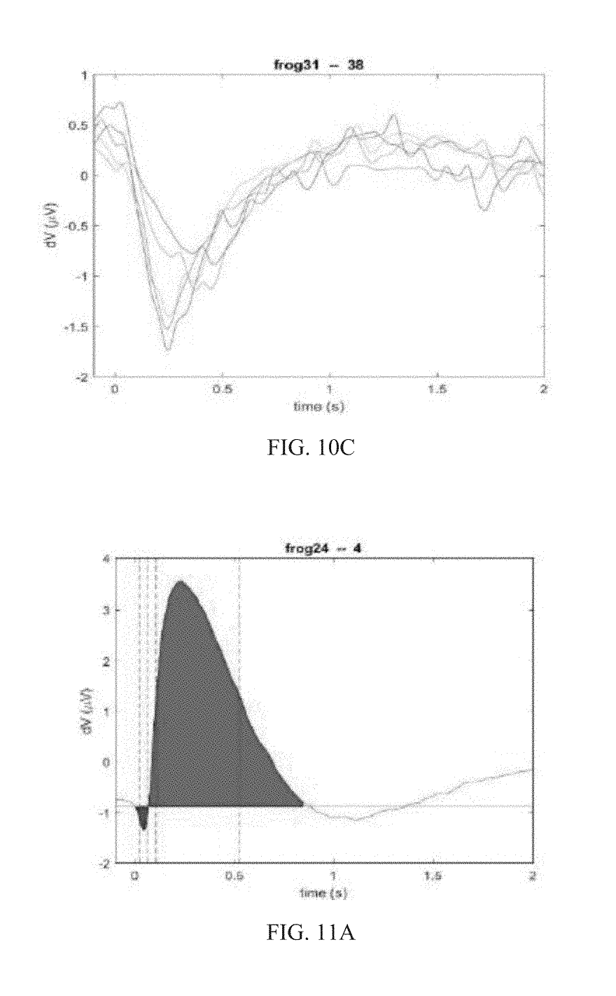

The “negative only” ERG was also seen primarily after short X-ray pulses to the retina, but these were also seen in control measurements where the electrodes were placed subcutaneously on the frog's back. FIGS. 10A-10C show a summary of the “negative only” x-ray ERG responses.

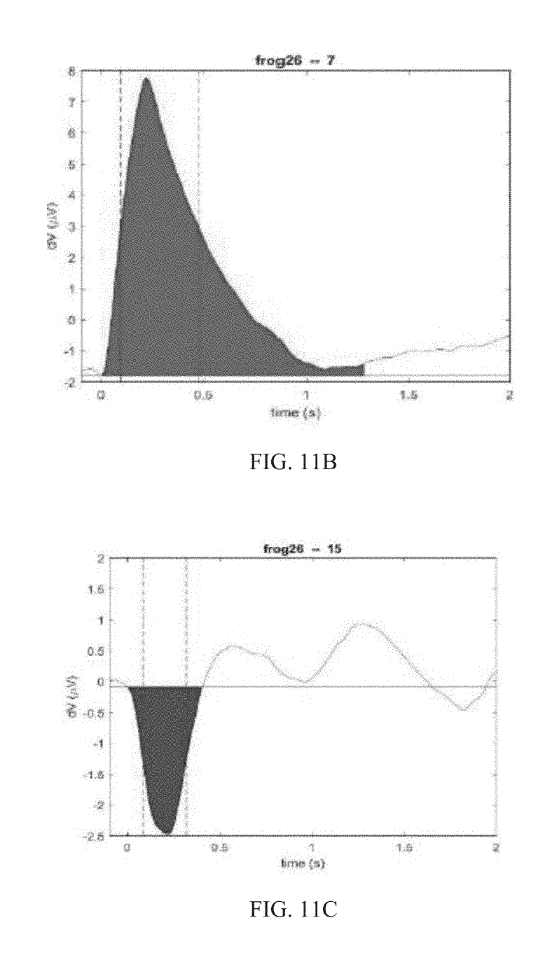

FIGS. 11A-11C show a summary of how FWHM and AUC were calculated for each of the response types. As seen in the scatter plots, the a-waves of the visible responses were clearly different from the negative responses to X-ray stimuli. The b-wave and the positive X-ray response distributions, on the other hand, were not separable by these metrics.

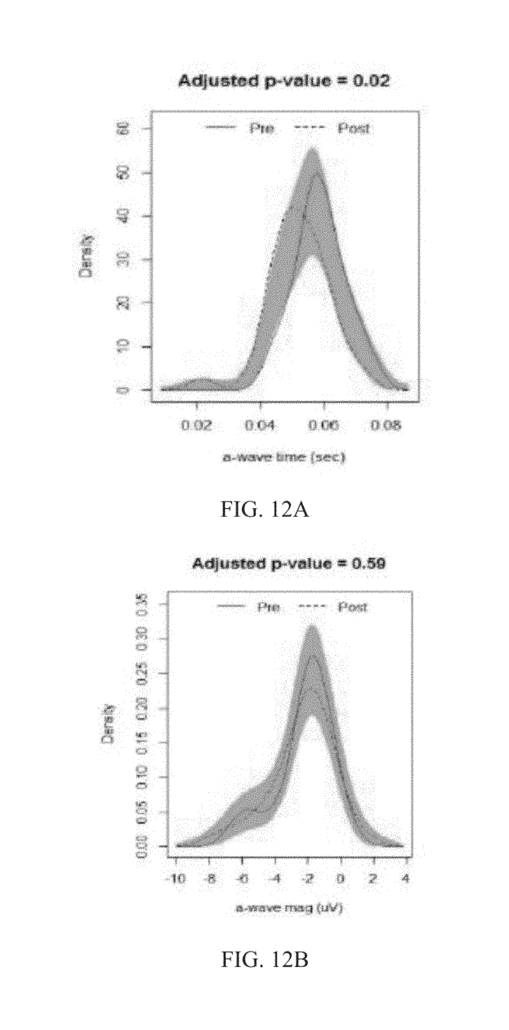

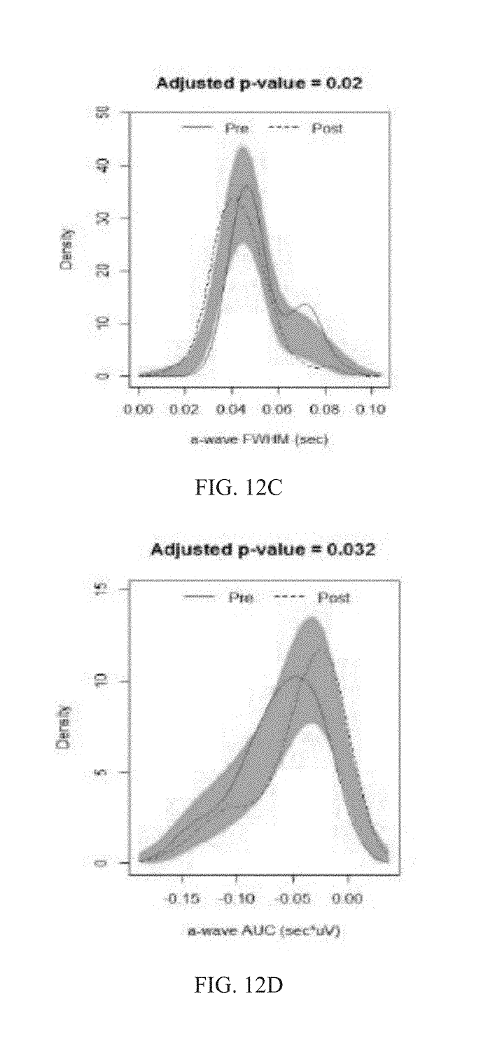

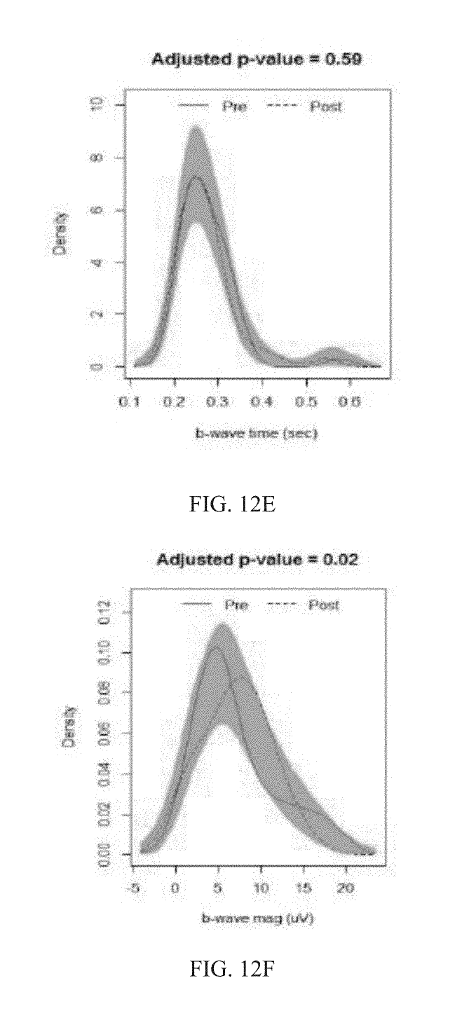

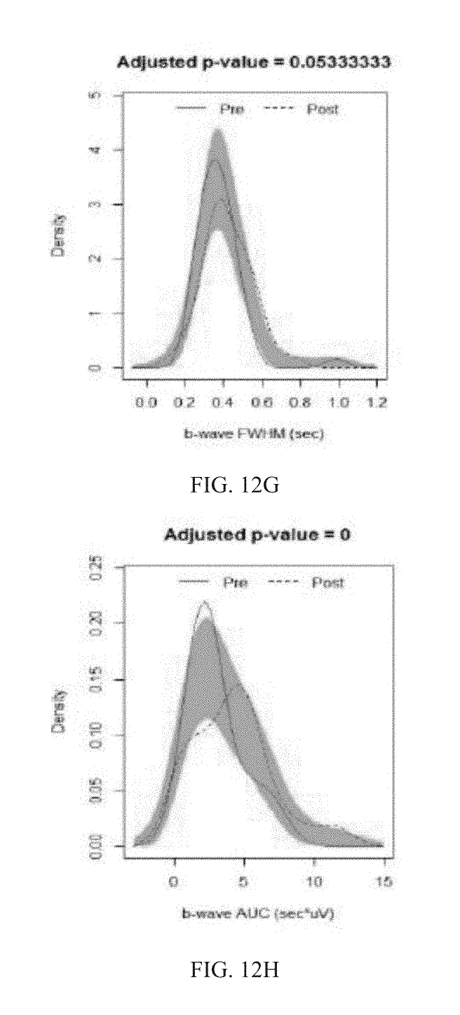

As a result of the low-power parametric models achieved by the experimental design, non-parametric models were used to draw conclusions about the feature-set. The visible responses were split into two groups based on whether or not the recordings were made before or after X-ray exposure. FIGS. 12A-12H show the non-parametric density distributions of these two groups for each feature (including AUC and FWHM measurements). The p-values output from the univariate sm.density.compare( ) function in R were adjusted using the Benjamini and Hochberg method ([113], which is hereby incorporated herein by reference in its entirety).

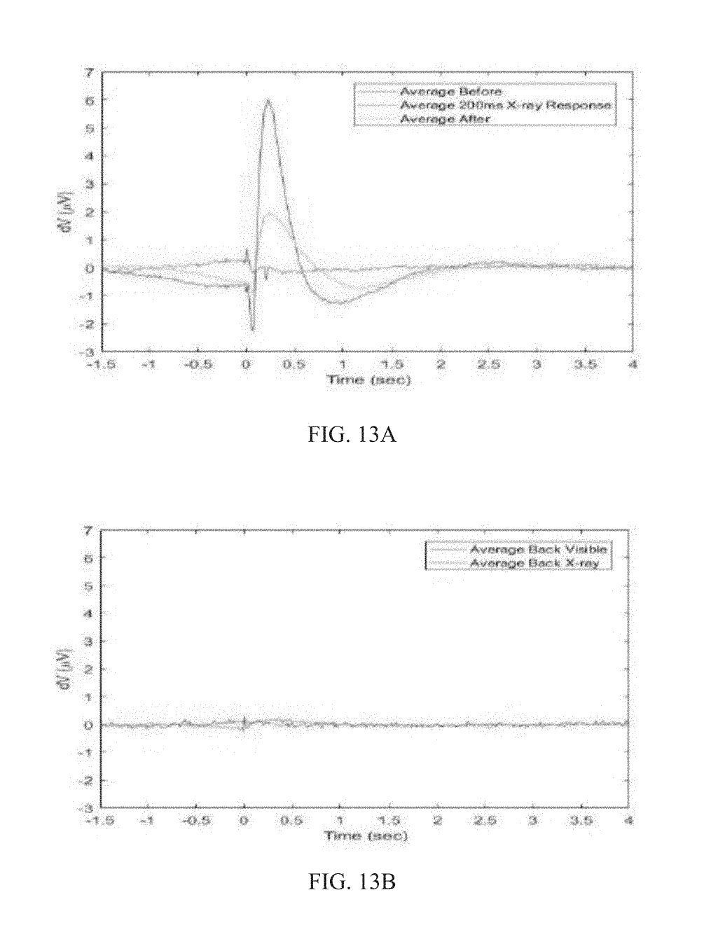

It was clear during the ERG acquisition process, and remained evident after the signal characterization and processing, that two subgroups of the frogs could be made based on their responses to the X-ray signal. The first subgroup of those that did not have eye ERG measurements that showed evidence of a direct retinal response to X-ray included four frogs (arbitrarily labeled 24, 25, 29, and 30). FIGS. 13A and 13B show the representative eye and back control responses, respectively, for frog 29. Despite the large decreases in a- and b-wave amplitudes seen for frog 29, the X-ray insensitive frogs, as a group, did not show significant differences in the feature distributions shown above.

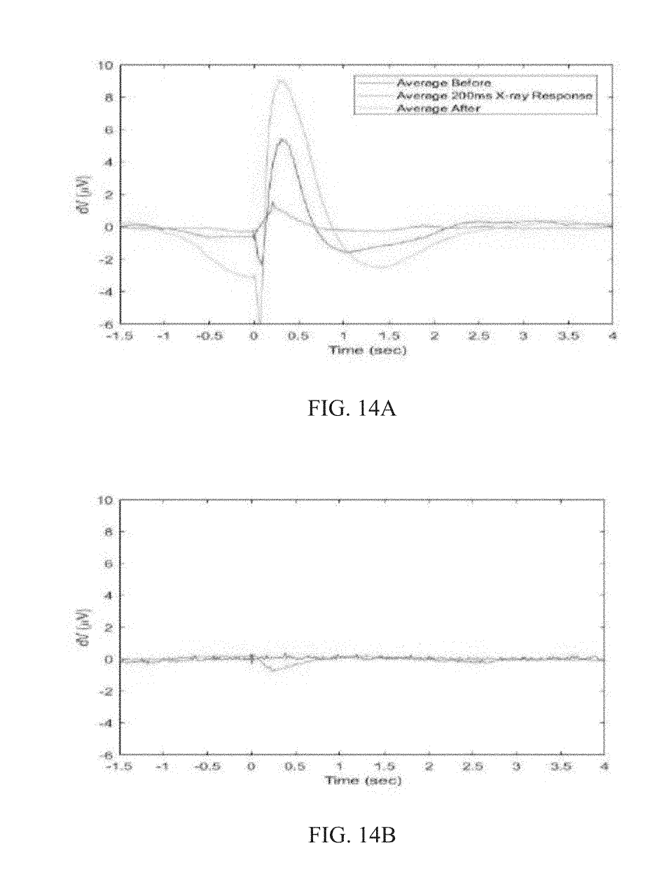

The other four frogs (arbitrarily labeled as 26, 27, 28, and 31) did show evidence of a direct retinal response to X-ray. Of these responses, all but three were in the positive direction and resembled the responses reported previously ([7]-[12]). The other three, all from frog 26, were negative (the direction of the a-wave), but had amplitudes that resembled those of small b-waves. FIGS. 14A and 14B shows the representative eye and back control responses, respectively, for frog 31. The differences seen in these figures are significant after the non-parametric distribution comparisons with significantly different features between the before and after groups in a-wave peak time, a-wave FWHM, a-wave AUC, and b-wave AUC. The b-wave FWHM had a p-value equal to 0.096.

Conventionally, the a-wave is negative and the b-wave is positive when the positive electrode is placed on the corneal surface or in the vitreous humor and the negative electrode is place on the other side of the retina with the surface of the retina running parallel to the electrodes. This convention, however, requires very precise and consistent placement of the electrodes. Also, the light sensitive rods and cones are well known to be differentially distributed across the surface of the retina. Therefore, it is possible (and this was observed) to place the electrodes as described and measure visible ERGs, (and X-ray responses), which were the opposite of the conventional polarity of a typical ERG. This discussion of polarity is one that is affected by both the distribution of sensitive cells and the geometry of the eye/retina with respect to the final electrode placements. Because of the ambiguity seen in ERG polarity, the ability to determine the legitimacy and relative polarity to visible ERG measurements is convoluted.

The mean a-/b-wave amplitudes were lower than what may be expected for amphibian ERG recordings. This could be due to the anesthesia given to the frogs before testing. Anesthetized frogs stop pulmonary respiration and only respire through their skin. However, in the experimental set-up described, there was no supplementary oxygen supplied to the frog. Therefore, it is likely that the frogs were being tested under hypoxic conditions, and hypoxia has been shown to reduce ERG amplitude by up to 75% in leopard frogs ([114]). In parallel to the diminished visible ERG responses, it may also follow that the retina's ability to respond to X-ray photons is also decreased. Nevertheless, the system was capable of measuring these minute signals with consistently high SNR. Increasing the response may not only give rise to higher visible responses, but also more definitive X-ray responses.

The non-parametric comparisons of the visible ERG before and after X-ray exposure demonstrate that repeated low-dose exposure of X-ray do have an effect on the retinal response to visible light regardless of whether there was a direct response to X-rays. Also, the significant effects seem to corroborate that the X-ray sensitivity stems from the rhodopsins as the largest effects were seen in the timing and duration of the rod-driven a-wave. Significant effects were also seen in the AUC of the b-wave.

Despite the differences seen between the before and after exposure groups, monitoring the effect of X-ray on the visible signal is merely an indirect measure of X-ray sensitivity. In addition to the comparisons made before and after exposure, a brief description of those recordings that showed a direct X-ray response was also provided.

### Example 2

A hybrid visible/X-ray ERG system was used to measure ERGs in 30 Northern leopard frogs. Dark-adapted frogs were anesthetized, placed in an animal holder, and subject to a sequence of light and X-ray pulses. In the experiments, X-rays were pulsed using a lead shutter controlled by a solenoid. Platinum electrodes were placed on the corneas of the frogs and behind their eyes in order to measure the differential potential changes induced by the pulse stimulation. While the frog was stimulated with visible light or X-ray pulses, the ERG signals were recorded on an AD Instruments device.

In the best controlled experiments, 8 frogs were tested for X-ray sensitivity. Of those frogs, three registered X-ray responses that were distinguishable from the background noise and drift. After responding to X-ray stimuli, an increase in visible light ERG was generally observed in these frogs. The total absolute area under the a, b, and c waves of the visible light ERG signals was used as an indicator for overall retinal response.

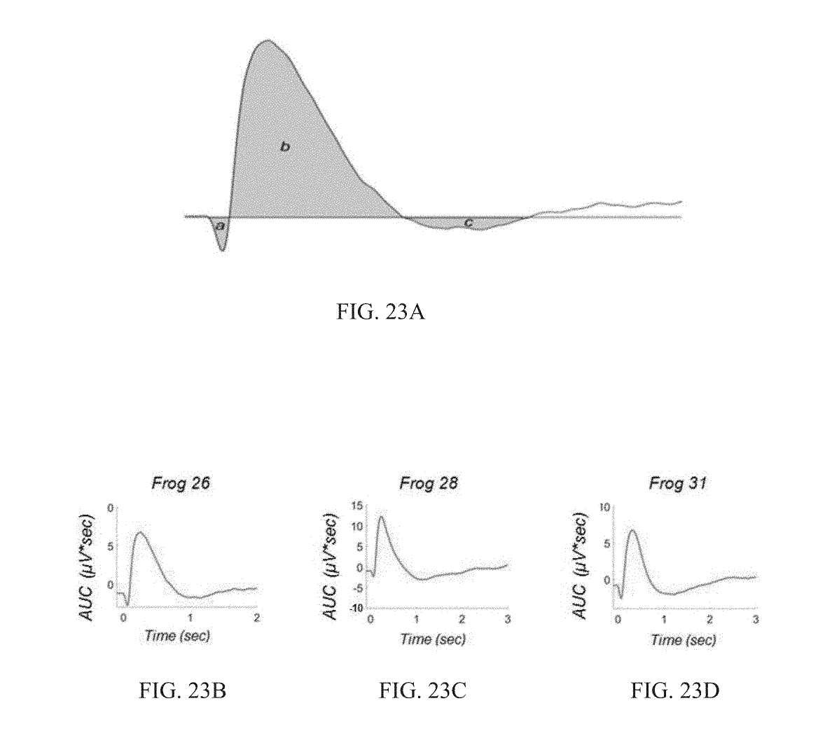

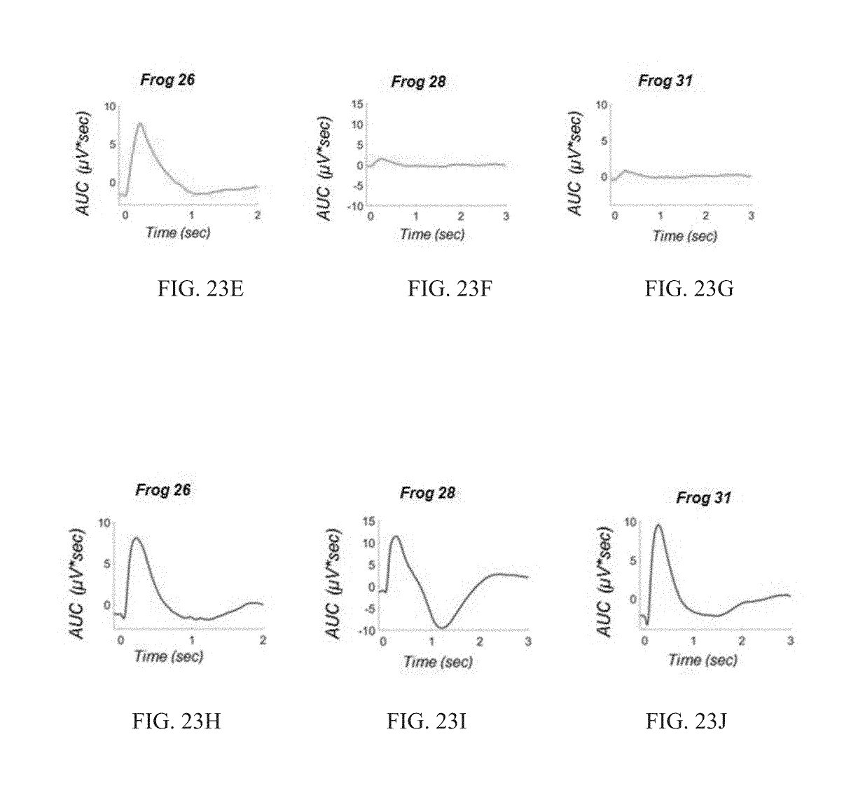

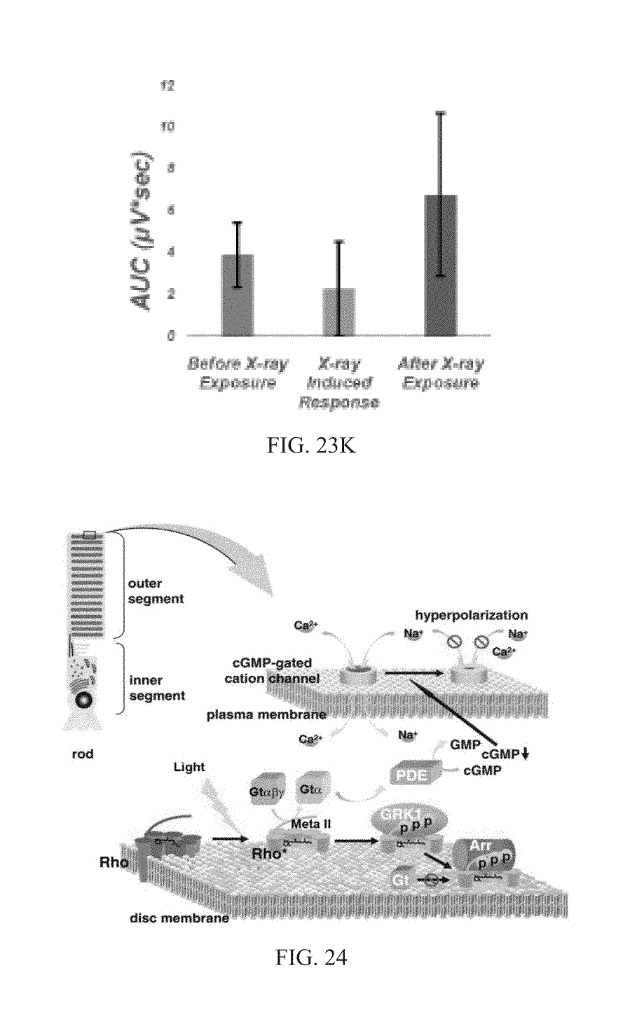

FIG. 23A shows the total absolute area under the curve of a, b, and c waves relative to the response voltage baseline, an indicator for overall retinal response. FIGS. 23B, 23C, and 23D show the visible light-induced ERGs before X-ray exposure for the three frogs, respectively, that registered an X-ray response (arbitrarily labeled as Frog 26, Frog 28, and Frog 31). FIGS. 23E, 23F, and 23G show the X-ray induced response for the same three frogs, respectively. FIGS. 23H, 23I, and 23J show the visible light-induced ERGs after X-ray exposure for the same three frogs, respectively. Each of the plots in FIGS. 23B-23J is in area under the curve (AUC, micro-Volts*second) versus time (seconds). FIG. 23K shows the average and standard deviations for the data in FIGS. 23B-23J. Referring to FIGS. 23A-23K, the total absolute area of the visible light ERG signals after X-ray exposure was larger than before X-ray exposure curves, suggesting X-ray exposure facilitates visible light phototransduction.

X-ray stimuli evoked a mono-phasic response (FIGS. 23E-23G), and X-ray exposure enhanced visible light-evoked ERG (FIGS. 23H-23K), indicating that X-ray appears to share the phototransduction pathways with visible light.

### Example 3

An image reconstruction demonstration with deep learning was performed. A poor-quality initial image was reconstructed to a good-quality image. A 2D world of Shepp-Logan phantoms was defined. A field of view was a unit disk covered by a 128*128 image, 8 bits per pixel. Each member image was one background disk of radius 1 and intensity 100 as well as up to 9 ellipses completely inside the background disk. Each ellipse was specified by the following random parameters: center at (x, y), axes (a, b), rotation angle q, and intensity selected from [−10, 10]. A pixel in the image could be covered by multiple ellipses including the background disk. The pixel value is the sum of all the involved intensity values. From each image generated, 256 parallel-beam projections were synthesized, 180 rays per projection. From each dataset of projections, a simultaneous algebraic reconstruction technique (SART) reconstruction was performed for a small number of iterations. This provided blurry intermediate images. Then, a deep network was trained using the known original phantoms to predict a much-improved image from a low-quality image. FIG. 20 shows the results of this demonstration. The image pair in the left-most column are two original phantoms; the image pair in the second-from-the-left column are the SART reconstruction after 20 iterations; the image pair in the second-from-the-right column are the SART reconstruction after 500 iterations; and the image pair in the right-most column are the deep imaging results after starting with the corresponding 20-iteration image (from the second-from-the-left column) as the inputs, which are very close to the 500-iteration images, respectively.

### Example 4

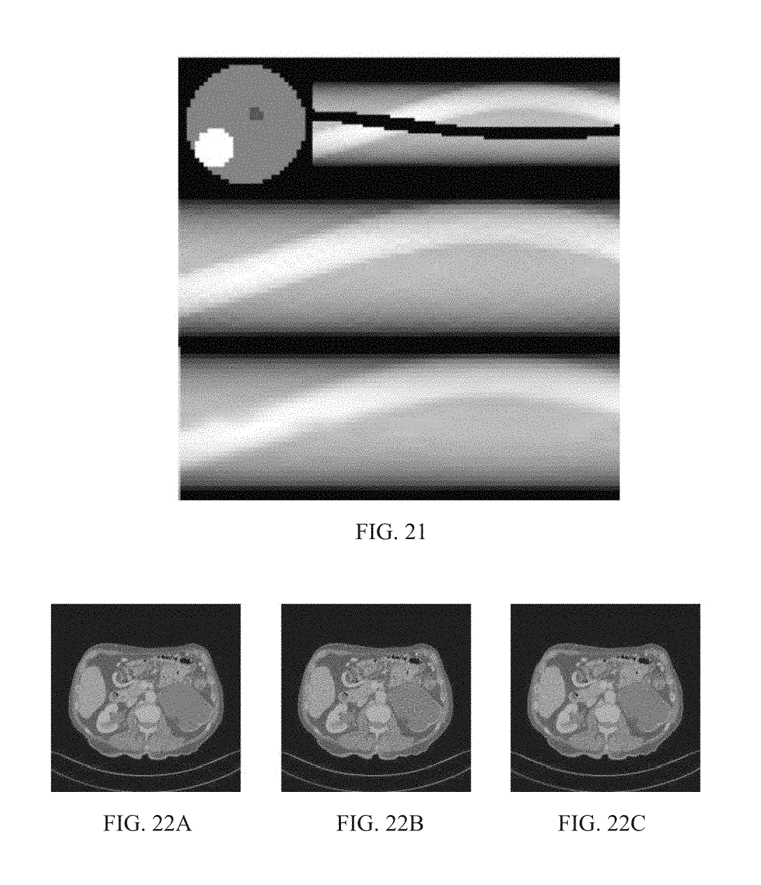

Another image reconstruction demonstration with deep learning was performed. A poor-quality sinogram was reconstructed to a good-quality sinogram, which was prepared in a way quite similar to that for Example 3. Each phantom contained a fixed background disk and two random disks inside the circular background; one disk represents an X-ray attenuating feature, and the other an X-ray opaque metal part. The image size was made 32×32 for quick results. After a phantom image was created, the sinogram was generated from 90 angles. Every metal-blocked sinogram was linked to a complete sinogram formed after the metal was replaced with an X-ray transparent counterpart. Then, a deep network was trained with respect to the complete sinograms to restore missing data. FIG. 21 shows the results of this demonstration. The first row shows the original image (metal is the small (purple) dot in the upper-left corner) and the associated metal-blocked sinogram. The second and third rows show the original and restored sinograms, respectively. Referring to FIG. 21, deep learning has much potential as a smart interpolator over missing data.

### Example 5

Another image reconstruction demonstration with deep learning was performed. FIGS. 22A-22C show the results of the experiment from the Mayo CT database (see also http://www.aapm.org/GrandChallenge/LowDoseCT). FIG. 22A shows the full-dose filtered back-projection image; FIG. 22B shows the quarter-dose filtered back-projection image; and FIG. 22C shows the deep learning reconstruction image using the quarter-dose filtered back-projection image of FIG. 22B as the starting input. Referring to FIGS. 22A-22C, the deep learning reconstruction image matches quite well with the full-dose filtered back-projection image.

It should be understood that the examples and embodiments described herein are for illustrative purposes only and that various modifications or changes in light thereof will be suggested to persons skilled in the art and are to be included within the spirit and purview of this application.

All patents, patent applications, provisional applications, and publications referred to or cited herein (including those in the “References” section) are incorporated by reference in their entirety, including all figures and tables, to the extent they are not inconsistent with the explicit teachings of this specification.

### REFERENCES

1. Zerhouni E. Medicine. The NIH roadmap. Science. 2003; 302(5642):63-72. doi: 10.1126/science.1091867. PubMed PMID: 14526066.
2. Insel T R, Landis S C, Collins F S. Research priorities. The NIH BRAIN initiative. Science. 2013; 340(6133):687-8. doi:10.1126/science.1239276. PubMed PMID: 23661744.
3. Deisseroth K. Optogenetics. Nat Methods. 2011; 8(1):26-9. doi: 10.1038/nmeth.f324. PubMed PMID: 21191368.
4. Fenno L, Yizhar O, Deisseroth K. The development and application of optogenetics. Annu Rev Neurosci. 2011; 34:389-412. doi: 10.1146/annurev-neuro-061010-113817. PubMed PMID: 21692661.
5. Jacques S L. Optical properties of biological tissues: a review. Phys Med Biol. 2013; 58(11):R37-61. doi: 10.1088/0031-9155/58/11/R37. PubMed PMID: 23666068.
6. Berry R, Getzin M, Gjesteby L, Wang G. X-optogenetics and u-optogenetics: Feasibility and possibilities. Photonics; 2015: Multidisciplinary Digital Publishing Institute.
7. Bachofer C S, Wittry S E. Electroretinogram in response to x-ray stimulation. Science. 1961; 133(3453):642-4. PubMed PMID: 13685657.
8. Bachofer C S, Wittry S E. Comparison of stimulus energies required to elicit the ERG in response to x-rays and to light.J Gen Physiol. 1962; 46:177-87. PubMed PMID: 13965919; PMCID: 2195264.
9. Bachofer C S, Wittry S E. Comparison of electroretinal response to x-rays and to light. Radiat Res. 1962; 17:1-10. PubMed PMID: 13863740.
10. Bachofer C S, Wittry S E. Interactions of x-rays and light in the production of the electroretinogram. Exp Eye Res. 1963; 2:141-7. PubMed PMID: 13965920.
11. Bachofer C S, Wittry S E. Immediate retinal response to x-rays at milliroentgen levels. Radiat Res. 1963; 18:246-54. PubMed PMID: 13965918.
12. Bachofer C S, Wittry S E. Off-response of electroretinogram induced by X-ray stimulation. Vision Research. 1963; 3(1-2):51-9. doi: Doi 10.1016/0042-6989(63)90067-1. PubMed PMID: WOS:A1963WV93700006.
13. Miller G. Optogenetics. Shining new light on neural circuits. Science. 2006; 314(5806):1674-6. doi:10.1126/science.314.5806.1674. PubMed PMID: 17170269.
14. Hososhima S, Yuasa H, Ishizuka T, Hoque M R, Yamashita T, Yamanaka A, Sugano E, Tomita H, Yawo H. Near-infrared (NIR) up-conversion optogenetics. Sci Rep. 2015; 5:16533. doi: 10.1038/srep16533. PubMed PMID: 26552717; PMCID: 4639720.
15. Kattnig D R, Solov'yov I A, Hore P J. Electron spin relaxation in cryptochrome-based magnetoreception. Phys Chem Chem Phys. 2016; 18(18):12443-56. doi: 10.1039/c5cp06731f. PubMed PMID: 27020113.
16. Niessner C, Denzau S, Stapput K, Ahmad M, Peichl L, Wiltschko W, Wiltschko R. Magnetoreception: activated cryptochrome 1a concurs with magnetic orientation in birds. J R Soc Interface. 2013; 10(88):20130638. doi:10.1098/rsif.2013.0638. PubMed PMID: 23966619; PMCID: 3785833.
17. Lin J Y, Knutsen P M, Muller A, Kleinfeld D, Tsien R Y. ReaChR: a red-shifted variant of channelrhodopsin enables deeptranscranial optogenetic excitation. Nature neuroscience. 2013; 16:1499-508.
18. Prigge M, Schneider F, Tsunoda S P, Shilyansky C, Wietek J, Deisseroth K, Hegemann P. Color-tuned channelrhodopsins for multiwavelength optogenetics. J Biol Chem. 2012; 287(38):31804-12. doi:10.1074/jbc.M112.391185. PubMed PMID: 22843694; PMCID: 3442514.
19. Airan R D, Thompson K R, Fenno L E, Bernstein H, Deisseroth K. Temporally precise in vivo control of intracellular signalling. Nature. 2009; 458(7241):1025-9. doi: 10.1038/nature07926. PubMed PMID: 19295515.
20. Bellucci B. A proposito della eccitazione retinica da parte die raggi Roentgen (The question of retinal stimulation by Roentgen rays). Gior Hal Of tal. 1951; 4:249.
21. Lipetz L E. The x ray and radium phosphenes. The British journal of ophthalmology. 1955; 39:577.
22. Gegear R J, Foley L E, Casselman A, Reppert S M. Animal cryptochromes mediate magnetoreception by an unconventional photochemical mechanism. Nature. 2010; 463(7282):804-7. doi: 10.1038/nature08719. PubMed PMID: 20098414; PMCID: 2820607.
23. Lipetz L E. Electrophysiology of the X-ray phosphene. Radiation Research. 1955; 2(4):306-29. PubMed PMID: WOS:A1955XD78900002.
24. Ely T S. X-rays are visible—Radiation phosphene. Journal of Occupational Medicine. 1968; 10(1):9-13. PubMed PMID: WOS:A1968ZD89700003.
25. Dawson W W, Wiederwohl H. Functional alteration of visual receptor units and retinal pigments by X-irradiation. Radiat Res. 1965; 24:292-304. PubMed PMID: 14282682.
26. Dawson W W. Adaptation to equivalent visible and high-energy quanta. Radiat Res. 1969; 38(2):425-36. PubMed PMID: 5771807.
27. Tobias C A, Budinger T F, Lyman J T. Radiation-induced light flashes observed by human subjects in fast neutron, x-ray and positive pion beams. Nature. 1971; 230(5296):596-8. PubMed PMID: 4928670.
28. Chaddock T E. Visual detection of x-ray by the rhesus monkey. J Comp Physiol Psychol. 1972; 78(2):190-201. PubMed PMID: 4621692.
29. Demirchoglian G G. On the effect of ionizing radiation upon the retina in man and animals. Life sciences and space research. 1972; 11:281-94.
30. Malachowski M J. Effects of ionizing radiation on the light sensing elements of the retina.[Structural and physiological effects of carbon, helium, and neon ions on rods and cones of salamanders and mice]. California Univ., Berkeley (USA). Lawrence Berkeley Lab., 1978.
31. Doly M, Isabelle D B, Vincent P, Gaillard G, Meyniel G. Mechanism of the formation of X-ray-induced phosphenes: I. Electrophysiological investigations. Radiation research. 1980; 82:93-105.
32. Doly M, Isabelle D B, Vincent P, Gaillard G, Meyniel G. Mechanism of the formation of X-ray-induced phosphenes: II. Photochemical investigations. Radiation research. 1980; 82:430-40.
33. Steidley K D, Eastman R M, Stabile R J. Observations of visual sensations produced by Cerenkov radiation from highenergy electrons. International Journal of Radiation Oncology*Biology*Physics. 1989; 17:685-90.
34. Nozdrachev A D, Savchenko B N. X-ray phosphene is an indicator of radiation excitability of the cns. Doklady Akademii Nauk. 1993; 329(1):106-9. PubMed PMID:WOS:A1993LG51000028.
35. Savchenko B N. Specific features of the electroretinogram of vertebrates induced by X-rays. Neuroscience and behavioral physiology. 1993; 23:49-55.
36. Nozdrachev A D, Zavarina L B, Savchenko B N. Light and X-ray electroretinogram evoked reactions at the different levels of retinal adaptation to light. Doklady Akademii Nauk. 1994; 334(1):118-20. PubMed PMID: WOS:A1994NA82500036.
37. Loizzo S, Guarino I, Brusa A, Fadda A, Loizzo A, Lopez L, Pedrazzo G, Capasso A. A neurophysiological approach to radiation-induced ‘phosphene’ phenomenon: Studies in awake and anaesthetized mice. Current Neurobiology. 2013; 4(1 & 2):47-52.
38. Wang G, Hoffman E A, McLennan G, Bohnenkamp F, Colliso F, Cong W X, Jiang M, Kumar D, Li H, Li Y, McCray P, Meinel J F, Ritman E, Suter M, Taft P, Tian J, Wang L H, Zabner J, Zhu F P. Development of the first bioluminescent C T scanner. Radiology. 2003; 229:566.
39. Wang G, Cong W X, Shen H O, Qian X, Henry M, Wang Y. Overview of bioluminescence tomography-a new molecular imaging modality. Frontiers in Bioscience-Landmark. 2008; 13:1281-93. doi: 10.2741/2761. PubMed PMID: WOS:000255775700105.
40. Wang G, Cong W X, Durairaj K, Qian X, Shen H, Sinn P, Hoffman E, McLennan G, Henry M. In vivo mouse studies with bioluminescence tomography. Optics Express. 2006; 14(17):7801-9. doi: Doi 10.1364/0e.14.007801. PubMed PMID: WOS:000240164100037.
41. Wang G, Li Y, Jiang M. Uniqueness theorems in bioluminescence tomography. Medical Physics. 2004; 31(8):2289-99. doi: 10.1118/1.1766420. PubMed PMID: WOS: 000223316600015.
42. Pfeiffer F, Weitkamp T, Bunk O, David C. Phase retrieval and differential phase-contrast imaging with low-brilliance Xray sources. Nature Physics. 2006; 2(4):258-61. PubMed PMID: ISI:000236979500016.
43. Chtcheprov P, Burk L, Yuan H, Inscoe C, Ger R, Hadsell M, Lu J, Zhang L, Chang S, Zhou O. Physiologically gated microbeam radiation using a field emission x-ray source array. Med Phys. 2014; 41(8):081705. doi: 10.1118/1.4886015. PubMed PMID: 25086515; PMCID: 4105967.
44. Liu B, Wang G, Ritman E L, Cao G, Lu J, Zhou O, Zeng L, Yu H. Image reconstruction from limited angle projections collected by multisource interior x-ray imaging systems. Phys Med Biol. 2011; 56(19):6337-57. doi: 10.1088/0031-9155/56/19/012. PubMed PMID: 21908905; PMCID: 3193606.
45. Ashton J R, West J L, Badea C T. In vivo small animal micro-C T using nanoparticle contrast agents. Front Pharmaco1.2015; 6:256. doi: 10.3389/fphar.2015.00256. PubMed PMID: 26581654; PMCID: 4631946.
46. Baldelli P, Taibi A, Tuffanelli A, Gambaccini M. Quasi-monochromatic x-rays for diagnostic radiology. Phys Med Biol. 2003; 48(22):3653-65. PubMed PMID: 14680265.
47. Banerjee S, Chen S Y, Powers N, Haden D, Liu C, Golovin G, Zhang J, Zhao B Z, Clarke S, Pozzi S, Silano J, Karwowski H, Umstadter D. Compact source of narrowband and tunable X-rays for radiography. Nuclear Instruments & Methods in Physics Research Section B-Beam Interactions with Materials and Atoms. 2015; 350:106-11. doi:10.1016/j.nimb.2015.01.015. PubMed PMID: WOS:000354342200020.
48. Ham K, Butler L G. Algorithms for three-dimensional chemical analysis via multi-energy synchrotron X-ray tomography. Nuclear Instruments & Methods in Physics Research Section B-Beam Interactions with Materials and Atoms. 2007; 262(1):117-27. doi: 10.1016/j.nimb.2007.04.300. PubMed PMID: WOS:000248778800019.
49. Baldwin W F, Sutherland J B. Extreme sensitivity to low-level X-rays in the eye of the cockroach Blaberus. Radiation Research. 1965; 24:513-8.
50. Gurtovoi G K, Burdianskaia Y O. Threshold reactivity of various regions of the human retina to X-irradiation. Biophysics (USSR)(English Translation). 1960; 5.
51. Hendy J H. Physical mechanisms in radiation biology. International Journal of Radiation Biology and Related Studies in Physics, Chemistry and Medicine. 1975; 27(1):103-.
52. Lange K. Mathematical and statistical methods for genetic analysis. 2nd ed. New York: Springer; 2002. xvii, 361 p. p.
53. Wilcox R R. Fundamentals of modern statistical methods: substantially improving power and accuracy. 2nd ed. New York, N.Y.: Springer; 2010. xvi, 249 p. p.
54. Baylor D A, Lamb T D, Yau K W. Responses of retinal rods to single photons. J Physiol. 1979; 288:613-34. PubMed PMID: 112243; PMCID: 1281447.
55. Baylor D A, Lamb T D, Yau K W. The membrane current of single rod outer segments. J Physiol. 1979; 288:589-611. PubMed PMID: 112242; PMCID: 1281446.
56. Forti S, Menini A, Rispoli G, Torre V. Kinetics of phototransduction in retinal rods of the newt Triturus cristatus. J Physiol. 1989; 419:265-95. PubMed PMID: 2621632; PMCID: 1190008.
57. Lamb T D, Baylor D A, Yau K W. The membrane current of single rod outer segments. Vision Res. 1979; 19(4):385. PubMed PMID: 112774.
58. Mazzolini M, Facchetti G, Andolfi L, Proietti Zaccaria R, Tuccio S, Treu J, Altafini C, Di Fabrizio E M, Lazzarino M, Rapp G, Torre V. The phototransduction machinery in the rod outer segment has a strong efficacy gradient. Proc Natl Acad Sci USA. 2015; 112(20):E2715-4. doi: 10.1073/pnas.1423162112. PubMed PMID: 25941368; PMCID: 4443333.
59. Rieke F, Baylor D A. Origin of reproducibility in the responses of retinal rods to single photons. Biophys J. 1998; 75(4):1836-57. doi: 10.1016/S0006-3495(98)77625-8. PubMed PMID: 9746525; PMCID: 1299855.
60. Sung C H, Chuang J Z. The cell biology of vision. Journal of Cell Biology. 2010; 190(6):953-63. doi: 10.1083/jcb.201006020. PubMed PMID: WOS:000282604600004.
61. Showell C, Conlon F L. Tissue sampling and genomic DNA purification from the western clawed frog Xenopus tropicalis. Cold Spring Harb Protoc. 2009; 2009(9):pdb prot5294. doi: 10.1101/pdb.prot5294. PubMed PMID: 20147279; PMCID: 3621791.
62. Burnett J C, Rossi J J. RNA-based therapeutics: current progress and future prospects. Chem Biol. 2012; 19(1):60-71. doi: 10.1016/j.chembio1.2011.12.008. PubMed PMID: 22284355; PMCID: 3269031.
63. Wald G, Brown P K. The molar extinction of rhodopsin. The Journal of general physiology. 1953; 37:189-200.
64. Hamm H E, Bownds M D. Protein complement of rod outer segments of the frog retina. Biochemistry. 1986; 25:4512-23.
65. Okada T, Matsuda T, Kandori H, Fukada Y, Yoshizawa T, Shichida Y. Circular dichroism of metaiodopsin II and its binding to transducin: a comparative study between meta II intermediates of iodopsin and rhodopsin. Biochemistry. 1994; 33:4940-6.
66. Okada T, Takeda K, Kouyama T. Highly selective separation of rhodopsin from bovine rod outer segment membranes using combination of divalent cation and alkyl(thio)glucoside. Photochem Photobiol. 1998; 67(5):495-9. PubMed PMID:9613234.
67. Morgan J E, Vakkasoglu A S, Lanyi J K, Gennis R B, Maeda A. Coordinating the structural rearrangements associated with unidirectional proton transfer in the bacteriorhodopsin photocycle induced by deprotonation of the proton-release group: a time-resolved difference FTIR spectroscopic study. Biochemistry. 2010; 49:3273-81.
68. H. Greenspan, B. V. Ginneken, and R. M. Summers, “Guest editorial. Deep Learning in Medical Imaging: Overview and Future Promise of an Exciting New Technique,” IEEE Trans Med Imaging, vol. 35, pp. 1153-1159, March 2016.
69. J. H. Byrne, R. Heidelberger, and M. N. Waxham, From molecules to networks: an introduction to cellular and molecular neuroscience, Third edition. ed. Amsterdam; Boston: Elsevier/AP, Academic Press is an imprint of Elsevier, 2014.
70. M. Anthony and P. L. Bartlett, Neural network learning: theoretical foundations. Cambridge; New York, N.Y.: Cambridge University Press, 1999.
71. G. E. Hinton, S. Osindero, and Y. W. Teh, “A fast learning algorithm for deep belief nets,” Neural Computation, vol. 18, pp. 1527-1554, July 2006.
72. D. Silver, A. Huang, C. J. Maddison, A. Guez, L. Sifre, G. van den Driessche, et al., “Mastering the game of Go with deep neural networks and tree search,” Nature, vol. 529, pp. 484-+, Jan. 28 2016.
73. Y. LeCun, Y. Bengio, and G. Hinton, “Deep learning,” Nature, vol. 521, pp. 436-44, May 28 2015.
74. K. Hornik, “Multilayer feedforward networks are universal approximators,” Neural Networks, vol. 2, pp. 359-366, 1989.
75. K. Hornik, “Approximation capabilities of multilayer feedforward networks,” Neural Networks, vol. 4, pp. 251-257, 1991.
76. A. Katsevich, “An improved exact filtered backprojection algorithm for spiral computed tomography,” Advances in Applied Mathematics, vol. 32, pp. 681-697, May 2004.
77. C. H. McCollough, G. H. Chen, W. Kalender, S. Leng, E. Samei, K. Taguchi, et al., “Achieving routine submillisievert CT scanning: report from the summit on management of radiation dose in CT,” Radiology, vol. 264, pp. 567-80, August 2012.
78. G. Wang, M. Kalra, V. Murugan, Y. Xi, L. Gjesteby, M. Getzin, et al., “Vision 20/20: Simultaneous CT-MRI—Next chapter of multimodality imaging,” Med Phys, vol. 42, pp. 5879-89, October 2015.
79. http://arxiv.org/abs/1410.3831.
80. https://arxiv.org/abs/1312.6199.
81. http://arxiv. or g/pdf/1412.1897v2.pdf
82. F. Natterer and F. Withbeling, Mathematical methods in image reconstruction. Philadelphia: Society for Industrial and Applied Mathematics, 2001.
83. G. T. Herman and R. Davidi, “On Image Reconstruction from a Small Number of Projections,” Inverse Probl, vol. 24, pp. 45011-45028, August 2008.
84. V. Estellers, J. P. Thiran, and X. Bresson, “Enhanced compressed sensing recovery with level set normals,” IEEE Trans Image Process, vol. 22, pp. 2611-26, July 2013.
85. https://biometry.nci.nih.gov/cdas/datasets/nlst/.
86 https://www.nlm.nih.gov/research/visible/visible_human.html.
87. M. G. Stabin, X. G. Xu, M. A. Emmons, W. P. Segars, C. Shi, and M. J. Fernald, “RADAR reference adult, pediatric, and pregnant female phantom series for internal and external dosimetry,” J Nucl Med, vol. 53, pp. 1807-13, November 2012.
88. https://arxiv.org/ftp/quant-ph/papers/0202/0202131.pdf
89. T. S. Kuhn, The structure of scientific revolutions. Chicago: University of Chicago Press, 1962.
90. J. Preston and T. S. Kuhn, Kuhn's The structure of scientific revolutions: a reader's guide. London; New York: Continuum, 2008.
91. A. J. G. Hey, The fourth paradigm: data-intensive scientific discovery, 2009.
92. A. Shademan, R. S. Decker, J. D. Opfermann, S. Leonard, A. Krieger, and P. C. Kim, “Supervised autonomous robotic soft tissue surgery,” Sci Transl Med, vol. 8, p. 337ra64, May 4 2016.
93. Pugh E N and Lamb T D, Amplification and Kinetics of the Activation Steps in Phototransduction. Biochimica Et Biophysica Acta, 1993. 1141(2-3): p. 111-149.
94. Syeda S, Patel A K, Lee T, and Hackam A S, Reduced photoreceptor death and improved retinal function during retinal degeneration in mice lacking innate immunity adaptor protein MyD88. Exp Neurol, 2015. 267: p. 1-12.
95. Tanimoto N, Sothilingam V, Kondo M, Biel M, Humphries P, and Seeliger M W, Electroretinographic assessment of rod-and cone-mediated bipolar cell pathways using flicker stimuli in mice. Sci Rep, 2015. 5: p. 10731.
96. Ando R, Noda K, Tomaru U, Kamoshita M, Ozawa Y, Notomi S, et al., Decreased proteasomal activity causes photoreceptor degeneration in mice. Invest Ophthalmol Vis Sci, 2014. 55(7): p. 4682-90.
97. Mao H, Seo S J, Biswal M R, Li H, Conners M, Nandyala A, et al., Mitochondrial oxidative stress in the retinal pigment epithelium leads to localized retinal degeneration. Invest Ophthalmol Vis Sci, 2014. 55(7): p. 4613-27.
98. Abd-El-Barr M M, Pennesi M E, Saszik S M, Barrow A J, Lem J, Bramblett D E, et al., Genetic Dissection of Rod and Cone Pathways in the Dark-Adapted Mouse Retina. Journal of Neurophysiology, 2009. 102(3): p. 1945-1955.
99. Jiang H, Lyubarsky A, Dodd R, Vardi N, Pugh E, Baylor D, et al., Phospholipase C beta 4 is involved in modulating the visual response in mice. Proc Natl Acad Sci USA, 1996. 93(25): p. 14598-601.
100. Nagel G, Brauner M, Liewald J F, Adeishvili N, Bamberg E, and Gottschalk A, Light activation of channelrhodopsin-2 in excitable cells of Caenorhabditis elegans triggers rapid behavioral responses. Curr Biol, 2005. 15(24): p. 2279-84.
101. Li X, Gutierrez D V, Hanson M G, Han J, Mark M D, Chiel H, et al., Fast noninvasive activation and inhibition of neural and network activity by vertebrate rhodopsin and green algae channelrhodopsin. Proc Natl Acad Sci USA, 2005. 102(49): p. 17816-21.
102 Ishizuka T, Kakuda M, Araki R, and Yawo H, Kinetic evaluation of photosensitivity in genetically engineered neurons expressing green algae light-gated channels. Neurosci Res, 2006. 54(2): p. 85-94.
103. Boyden E S, Zhang F, Bamberg E, Nagel G, and Deisseroth K, Millisecond-timescale, genetically targeted optical control of neural activity. Nat Neurosci, 2005. 8(9): p. 1263-8.
104. Nagel G, Szellas T, Kateriya S, Adeishvili N, Hegemann P, and Bamberg E, Channelrhodopsins: directly light-gated cation channels. Biochem Soc Trans, 2005. 33(Pt 4): p. 863-6.
105. Bachofer C S and Wittry S E, Comparison of Stimulus Energies Required to Elicit Erg in Response to X-Rays and to Light. Journal of General Physiology, 1962. 46(2): p. 177-&.
106. Bachofer C S and Wittry S E, Interactions of x-rays and light in the production of the electroretinogram. Exp Eye Res, 1963. 2: p. 141-7.
107. Bachofer C S and Wittry S E, Immediate retinal response to x-rays at milliroentgen levels. Radiat Res, 1963. 18: p. 246-54.
108. Bachofer C S and Wittry S E, Electroretinogram in response to x-ray stimulation. Science, 1961. 133(3453): p. 642-4.
109. Bachofer C S and Wittry S E, Comparison of electroretinal response to x-rays and to light. Radiat Res, 1962. 17: p. 1-10.
110. Schmeling F, Wakakuwa M, Tegtmeier J, Kinoshita M, Bockhorst T, Arikawa K, et al., Opsin expression, physiological characterization and identification of photoreceptor cells in the dorsal rim area and main retina of the desert locust, Schistocerca gregaria. J Exp Biol, 2014. 217(Pt 19): p. 3557-68.
111.Bachofer C S and Wittry S E, Off-Response of Electroretinogram Induced by X-Ray Stimulation. Vision Research, 1963. 3(1-2): p. 51-59.
112.Sharma R, Sharma S D, Pawar S, Chaubey A, Kantharia S, and Babu D A, Radiation dose to patients from X-ray radiographic examinations using computed radiography imaging system. J Med Phys, 2015. 40(1): p. 29-37.
113.Benjamini Y and Hochberg Y, Controlling the false discovery rate: a practical and powerful approach to multiple testing. Journal of the royal statistical society Series B (Methodological), 1995: p. 289-300.
114. Stenslokken K-O, Milton S L, Lutz P L, Sundin L, Renshaw G M, Stecyk J A, et al., Effect of anoxia on the electroretinogram of three anoxia-tolerant vertebrates. Comparative Biochemistry and Physiology Part A: Molecular & Integrative Physiology, 2008. 150(4): p. 395-403.
115. Wang et al., International Patent Application Publication No. WO2014/176328.
116. Wang et al., U.S. Pat. No. 8,862,206.
117. Wang et al., U.S. Pat. No. 8,811,700.
118. Wang et al., U.S. Patent Application Publication No. 2011/0105880.
119. Wang et al., U.S. Pat. No. 7,697,658.
120. Wang et al., International Patent Application Publication No. WO2016/106348.
121. Wang et al., U.S. Patent Application Publication No. 2015/0157286.
122. Wang et al., U.S. Patent Application Publication No. 2015/0170361.
123. Wang et al., U.S. Patent Application Publication No. 2015/0193927.
124. Wang et al., International Patent Application Publication No. WO2015/164405.
125. Wang et al., U.S. Patent Application Publication No. 2016/0113602.
126. Wang et al., U.S. Patent Application Publication No. 2016/0135769.
127. Wang et al., U.S. Patent Application Publication No. 2016/0166852.
128. Wang et al., International Patent Application Publication No. WO2016/106348.
129. Wang et al., International Patent Application Publication No. WO2016/118960.
130. Wang et al., International Patent Application Publication No. WO2016/154136.
131. Wang et al., International Patent Application Publication No. WO2016/197127.
132. Wang et al., International Patent Application Publication No. WO2017/015381.
133. Wang et al., International Patent Application Publication No. WO2017/019782.
134. Wang et al., International Patent Application Publication No. WO2017/048856.
135. Wang et al., International Patent Application No. PCT/US2016/061890.
136. Wang et al., International Patent Application No. PCT/US2017/026322.
137. Wang et al., International Patent Application No. PCT/US2017/018456.
138. Wang et al., International Patent Application No. PCT/US2017/034011.
139. Maeda T, Imanishi Y, Palczewski K. Rhodopsin phosphorylation: 30 years later. Prog Retin Eye Res. 2003; 22(4):417-34. PubMed PMID: 12742390.
140. Chen C K. RGS Protein Regulation of Phototransduction. Prog Mol Biol Transl Sci. 2015; 133:31-45. doi: 10.1016/bs.pmbts.2015.02.004. PubMed PMID: 26123301; PMCID: PMC4664578.

1. A method of controlling the behavior of a neuron in a sample, the method comprising:
providing X-ray radiation to an X-ray sensitive biomolecule within the sample to stimulate the X-ray sensitive biomolecule,
wherein the stimulation of the X-ray sensitive biomolecule causes a change in the membrane potential of the neuron, thereby changing the behavior of the neuron.

2. The method according to claim 1, wherein the X-ray sensitive biomolecule is a protein.

3. The method according to claim 1, wherein the X-ray sensitive biomolecule is an opsin.

4. The method according to claim 1, wherein the X-ray sensitive biomolecule is a rhodopsin.

5. The method according to claim 1, wherein the X-ray sensitive biomolecule is channelrhodopsin, bacteriorhodopsin, or archaerhodopsin.

6. The method according to claim 1, wherein the X-ray sensitive biomolecule is rhodopsin from outer segments of rod cells in a retina.

7. The method according to claim 6, wherein the retina is a Northern leopard frog retina.

8. The method according to claim 1, wherein the sample is a living organism.

9. The method according to claim 8, wherein the sample is a Northern leopard frog.

10. The method according to claim 8, wherein the sample is a human.

11. The method according to claim 1, further comprising genetically modifying the neuron prior to providing the X-ray radiation to the X-ray sensitive biomolecule.

12-20. (canceled)

21. The method according to claim 11, wherein the X-ray sensitive biomolecule is a protein.

22. The method according to claim 11, wherein the X-ray sensitive biomolecule is an opsin.

23. The method according to claim 11, wherein the X-ray sensitive biomolecule is a rhodopsin.

24. The method according to claim 11, wherein the X-ray sensitive biomolecule is channelrhodopsin, bacteriorhodopsin, or archaerhodopsin.

25. The method according to claim 11, wherein the X-ray sensitive biomolecule is rhodopsin from outer segments of rod cells in a retina.

26. The method according to claim 25, wherein the retina is a Northern leopard frog retina.

27. The method according to claim 11, wherein the sample is a living organism.

28. The method according to claim 27, wherein the sample is a Northern leopard frog.

29. The method according to claim 27, wherein the sample is a human.
